(19) United States
(12) Patent Application Publication
(10) Pub. No.: US 2005/0028235 A1
(43) Pub. Date: Feb. 3, 2005

(54) Plant fruit with elevated potassium levels

(76) Inventors: Hong-Xia Zhang, Ontario (CA); Eduardo Blumwald, Davis, CA (US)

Correspondence Address: MORRISON & FOERSTER LLP, 425 MARKET STREET, SAN FRANCISCO, CA, 94105-2482, US

(21) Appl. No.: 10/617,623

(22) Filed: Jul. 10, 2003

Related U.S. Application Data
(60) Provisional application No. 60/395,637, filed on Jul. 12, 2002.

(51) Int. Cl. A01H 1/00; C12N 15/82

(52) U.S. Cl. 800/289

(57) ABSTRACT

The subject invention includes fruits with elevated potassium and the non-naturally salt tolerant occurring plants that produce fruit with elevated potassium when cultivated under elevated salt conditions. The present invention also includes methods of making such fruits and plants. One preferred method is generating a transgenic plant that has an ectopically expressed NHX related gene product.

### RELATED APPLICATIONS

This application claims the benefit of U.S. Provisional Application No. 60/395,637, filed Jul. 12, 2002, which is incorporated by reference herein in its entirety.

### FIELD OF INVENTION

This invention is in the field of agricultural biotechnology. In particular, this invention relates to non-naturally occurring plants that produce fruit with elevated potassium levels when grown under conditions of elevated salt.

### BACKGROUND OF THE INVENTION

Potassium is an important part of the human diet. Potassium in the diet has been shown to be beneficial to human health in a number of areas. In a recent study, people with the lowest levels of potassium in their diets were found to be 1.5 times more likely to suffer from strokes than people with the highest levels (Apr. 13, 1998, Journal of Neurology). Increases in potassium levels in people with low potassium diets were correlated with lowered blood pressure (July 2001, Journal of Hypertension). Furthermore, diuretics may cause a person to lose potassium thus heightening the need for additional potassium in the diet. Too little potassium can negatively impact muscle tissue, especially the heart. Thus, there is a need to produce foods that have increased potassium levels.

Salt sensitive plants when grown under elevated salt conditions produce fruit with elevated levels of potassium. However, the potassium level falls off near the time of harvest and growing salt sensitive plants under elevated salt conditions involves some difficulty because the plant will die if the salt levels are too high. By contrast, naturally salt tolerant plants grown under elevated salt conditions produce fruit with levels of potassium similar to the levels produced in fruit grown under low salt. (Maria C. Bolin, et al. Plant Science 160 (2001) 1153) Thus there is a need for plants that can be grown under high salt conditions and yet still produce fruit with elevated levels of potassium.

In addition, agricultural productivity is severely affected by soil salinity, and the damaging effects of salt accumulation in agricultural soils have influenced ancient and modern civilizations. Much research is aimed toward the breeding of crop cultivars with improved salt tolerance. One school of thought has concluded that salt tolerance will be achieved only after pyramiding several characteristics in a single genotype, where each one alone could not confer a significant increase in salt tolerance. (Yeo, et al. (1988) and Cuartero, et al. (1999)) (Full citations for the references cited herein are found after the Examples.) Arguably, salt tolerance is a complex trait, and the long list of salt stress-responsive genes seems to support this. (Zhu(2000)) The detrimental effects of salt on plants are a consequence of both a water deficit resulting in osmotic stress and the effects of excess sodium ions on key biochemical processes. In order to tolerate high levels of salts, plants should be able to utilize ions for osmotic adjustment and to internally distribute these ions to keep sodium away from the cytosol. There is thus a further need to produce salt tolerant plants. It would be particularly advantageous if the salt tolerant plants could produce fruit with elevated potassium levels since potassium is a key nutritional element as discussed above

### SUMMARY OF THE INVENTION

In order to meet these needs, the present invention is directed to transgenic fruit trees, berry plants, vines and vegetables that are able to grow and produce fruit with elevated potassium levels in the presence of elevated salt concentrations. In particular, the present invention is directed to salt tolerant tomato plants that produce tomatoes with elevated potassium levels.

In one aspect, the invention is directed to a non-naturally occurring plant or plant part from said plant comprising a fruit having increased potassium levels when said plant is cultivated under elevated salt conditions. In one variations, the increased potassium levels may be at least 10% higher, at least 15% higher, at least 20% higher, at least 25% higher, at least 30% higher, at least 35% higher, at least 40% higher, at least 45% higher, or at least 50% higher. In another variation, the cultivation under elevated salt conditions may be cultivation where the elevated salt conditions persist through the entire life cycle of the plant, the germination stage, the vegetative growth stage, the flowering stage, the seed embryogenesis stage, the stage of seed ripening, and any combination of the foregoing stages. In yet another variation, the fruit may be a flower developed fruit, an ovary developed fruit, a tomato, a grape, a strawberry, a peach, or an apple.

In another aspect, the non-naturally occurring salt tolerant plant comprises a transgene. In one variation, the transgene comprises a first nucleic acid sequence encoding a Na+/H+ transporter or a plant derived Na+/H+ transporter. In another variation, the transgene comprises a first nucleic acid selected from the following group: a nucleic acid molecule of the coding strand shown in SEQ ID NO:1, or a complement thereof; a nucleic acid molecule encoding the amino acid sequence shown in SEQ ID NO:2; a nucleic acid molecule that hybridizes to the sequence set forth in SEQ ID NO:1 or the complement of the sequence set forth in SEQ ID NO:1 under highly stringent conditions that include at least one wash in 0.1×SSC, 0.1% SDS, at 65° C. for thirty minutes; and a nucleic acid molecule encoding a plant NHX transporter polypeptide that hybridizes to the sequence set forth in SEQ ID NO:1 or the complement of the sequence set forth in SEQ ID NO:1 under moderately stringent conditions that includes at least one wash in 0.1×SSC, 0.1% SDS, at 50° C. for thirty minutes. In still another variation, the transgene further comprises a promoter sequence operably linked to the first nucleic acid sequence. In yet another variation, the promoter is a constitutive promoter or an inducible promoter. In certain variations, the promoter may be selected from the group consisting of the 35 S promoter and the CaMV promoter.

Another aspect of the present invention is a transgenic tomato comprising a first nucleic acid sequence selected from the group consisting of a nucleic acid molecule of the coding strand shown in SEQ ID NO:1, or a complement thereof; a nucleic acid molecule encoding the amino acid sequence shown in SEQ ID NO:2; a nucleic acid molecule that hybridizes to the sequence set forth in SEQ ID NO:1 or the complement of the sequence set forth in SEQ ID NO:1 under highly stringent conditions that include at least one wash in 0.1×SSC, 0.1% SDS, at 65° C. for thirty minutes; and a nucleic acid molecule encoding a plant NHX transporter polypeptide that hybridizes to the sequence set forth in SEQ ID NO:1 or the complement of the sequence set forth in SEQ ID NO:1 under moderately stringent conditions that includes at least one wash in 0.1×SSC, 0.1% SDS, at 50° C. for thirty minutes.

An additional aspect of the present invention is a seed produced from any of the foregoing plants and variations thereof.

The present invention also includes methods of generating the foregoing. One variation includes transfecting a plant with a transcriptional regulatory element and identifying salt tolerant plants comprising a fruit having increased potassium levels when said plant is cultivated under elevated salt conditions. In another variation, plants are transfected with a transcriptional regulatory element and identifying a plant wherein said transcriptional regulatory element has integrated operably linked to a Na+/H+ transporter. In yet another variation, the transcriptional regulatory element is a promoter, an enhancer element, a repressor element or a boundary element. In one variation, plants are transfected with a transgene comprising a Na+/H+ transporter and a salt tolerant plant comprising a fruit having increased potassium levels when said plant is cultivated under elevated salt conditions is identified. In one variation, the Na+/H+ transporter gene is selected from the group consisting of a nucleic acid molecule of the coding strand shown in SEQ ID NO:1, or a complement thereof; a nucleic acid molecule encoding the amino acid sequence shown in SEQ ID NO:2; a nucleic acid molecule that hybridizes to the sequence set forth in SEQ ID NO:1 or the complement of the sequence set forth in SEQ ID NO:1 under highly stringent conditions that include at least one wash in 0.1×SSC, 0.1% SDS, at 65° C. for thirty minutes; and a nucleic acid molecule encoding a plant NHX transporter polypeptide that hybridizes to the sequence set forth in SEQ ID NO:1 or the complement of the sequence set forth in SEQ ID NO:1 under moderately stringent conditions that includes at least one wash in 0.1×SSC, 0.1% SDS, at 50° C. for thirty minutes.

### BRIEF DESCRIPTION OF THE DRAWINGS

FIG. 1. Salt tolerance of wild-type tomato plants and transgenic plants overexpressing AtNHX1 grown in the presence of 200 mM NaCl. (A) wild-type plants grown in the presence of 5 mM NaCl. (B) transgenic plants overexpressing AtNHX1, grown in the presence of 5 mM NaCl. (C) Western blots from leaf membrane proteins (5 μg) tested with antibodies raised against AtNHX1. Upper panel: Lanes 1 and 4, tonoplast-enriched fraction; lanes 2 and 5, Golgi/ER-enriched fractions; 3 and 6, plasma membrane fraction. Lanes 1,2,3 correspond to membranes from wild-type plants while lanes 4,5,6 correspond to membranes from transgenic plants. Relative molecular masses are indicated on the left; lower panel: Enrichment of the fractions with tonoplast membranes was assessed with antibodies raised against the vacuolar H⁺-PPᵢase. (D) wild-type plants grown in the presence of 200 mM NaCl. (E)) transgenic plants overexpressing AtNHX1, grown in the presence of 200 mM NaCl. Plants shown after 11 weeks of growth.

Bar=25 cm.

FIG. 2. Na+/H+ exchange activity in leaf tonoplast vesicles Membrane fractions were purified from leaves using the method described with the modifications described. (Blumwald, et al. (1985) and Apse, et al. (1999)) At the indicated times, the vacuolar H+-PPᵢase was activated by the addition of Mg²⁺. When a steady-state pH gradient (acidic inside) was formed, the PPᵢ-dependent H⁺ transport activity was stopped by the addition of AMDP and the rates of cation/H⁺ exchange were determined in vesicles isolated from wild-type plants (WT) and transgenic plants overexpressing AtNHX1 (X1OE). (A) Na⁺-dependent H⁺ exchange, (B) K⁺-dependent H⁺ exchange. The addition of monensin (mon), an artificial Na⁺/H⁺ antiport, or nigericin (nig), an artificial K⁺/H⁺ antiport, abolished the pH gradient and the fluorescence was fully recovered. The figure shows a typical recording.

FIG. 3. Ion, sugar, and proline contents of wild-type and transgenic plants grown at different salt concentrations. Wild-type (hatched line bars) and transgenic plants (cross-hatched line bars) grown in the presence of 5 mM NaCl. Two independent transgenic lines (black and white bars) grown in the presence of 200 mM NaCl. (A) Na⁺ contents; (B) K⁺ contents; (C) Cl³¹ contents; (D) soluble sugar contents; (E) proline contents. For each determination, leaves, roots and fruits from ten plants were collected from each hydroponic tank and pooled. Values are the Mean ±S.D. from material collected from three hydroponic tanks (n=3).

FIG. 4. Fruits from wild-type and transgenic plants. (A) tomato fruits from wild-type plants; (B) tomato fruits from transgenic plants. (C) Western blots from fruit tonoplast proteins (5 μg) tested with antibodies raised against AtNHX1. Wild-type plants grown in the presence of 5 mM NaCl (lane 1). Two independent transgenic lines grown in the presence of 200 mM NaCl (lanes 2 and 3).

### DETAILED DESCRIPTION OF THE INVENTION

The present invention provides a non-naturally occurring fruit or vegetable producing plant that is characterized by producing fruit of increased potassium content. A preferred method of making such fruit or vegetable producing plant is to ectopically express a nucleic acid molecule encoding an NHX related gene product and cultivate the plant under elevated salt conditions. The NHX related gene product can have, for example, substantially the amino acid sequence of an NHX ortholog such as those described in Table II.

In one embodiment, the invention provides a transgenic fruit or vegetable producing plant characterized by producing fruit of elevated potassium content. A preferred method of producing such plant is by ectopic expression of an exogenous nucleic acid molecule encoding an NHX-related gene product. The nucleic acid molecule encoding the NHX-related gene product can be operatively linked to an exogenous regulatory element such as a constitutive regulatory element or a root, leaf or fruit-selective regulatory element.

The present invention is directed to the surprising discovery that NHX-1 regulates potassium levels in plant fruit. As disclosed herein, transgenic tomato plants over expressing an AtNHX1 were able to grow, flower and produce fruit with elevated potassium levels in the presence of 200 mM NaCl.

As further disclosed herein, overexpression of AtNHX1 in tomato results in the production of fruit having elevated potassium levels as compared to the fruit produced by wild type tomato. As set forth in the Example constitutive expression of NHX1 under control of a 35S promoter resulted in fruit having potassium levels about 120% the amount of potassium produced in fruit of wild type plants. In view of the isolation of NHX orthologs, as detailed in Table 2, the skilled artisan will recognize that an NHX related gene product, such as an ortholog of NHX, can also be used in the methods of the present invention, for example, to produce transgenic plants having the characteristics disclosed herein. Thus, the invention provides a non-naturally occurring fruit or vegetable and plants capable of producing the same such as a transgenic tomato plant, characterized by producing fruit with elevated potassium levels due to ectopic expression of a nucleic acid molecule encoding an NHX related gene product.

The term “plant fruit,” when used herein, refers to both the ovary developed fruit and the flower developed fruit. An “ovary developed fruit” is the developed ovary of a seed plant with its contents and accessory parts, as the pea pod, nut, tomato, pineapple, etc. A “flower developed fruit” is the edible part of a plant developed from a flower with any accessory tissues, as the peach, mulberry, banana, etc.

The term “elevated salt conditions,” when used herein, refers to a salinity level above the highest level at which a naturally occurring plant variety can thrive and produce fruit. It is recognized that the salt tolerance of plants varies between varieties. As used herein, the naturally occurring plant variety is understood to be the same plant variety as the non-naturally occurring plant variety but for the human introduced change. One of skill in the art understands that there can be natural variation in the salt tolerance of fruit producing plants even within a variety. Thus, elevated salt conditions are those conditions above which none of a particular variety can thrive and produce fruit. Determination of elevated salt conditions is routine and in many cases for commercially relevant crop already known.

As used herein, the term “non-naturally occurring,” when used in reference to a fruit or vegetable producing plant, means a seed plant that has been genetically modified by human intervention. A transgenic fruit or vegetable producing plant of the invention, for example, is a non-naturally occurring plant that contains an exogenous nucleic acid molecule, such as a nucleic acid molecule encoding an NHX related gene product and, therefore, has been genetically modified by human intervention. In addition, a seed plant that contains, for example, a mutation in an endogenous NHX related gene product regulatory element or coding sequence as a result of calculated exposure to a mutagenic agent, such as a chemical mutagen, or an “insertional mutagen,” such as a transposon, also is considered a non-naturally occurring seed plant, since it has been genetically modified by human intervention. Furthermore, a plant generated by cross breeding different strains and varieties are also considered a “non-naturally occurring plant,” because the selection and breeding is performed by human intervention. In contrast, a plant containing only spontaneous or naturally occurring mutations is not a “non-naturally occurring fruit or vegetable producing plant” as defined herein and, therefore, is not encompassed within the invention. One skilled in the art understands that, while a non-naturally occurring plant typically has a nucleotide sequence that is altered as compared to a similar naturally occurring seed plant, a non-naturally occurring plant also can be genetically modified by human intervention without altering its nucleotide sequence, for example, by modifying its methylation pattern.

Based upon the above definitions, it will be clear that a “non-naturally occurring salt tolerant plant” is a plant variety that has been genetically modified by human intervention and is capable of thriving and producing fruit at elevated salt conditions, i.e., at a salinity level above which a naturally occurring plant of the same variety cannot thrive and produce fruit.

The term “ectopically,” as used herein in reference to expression of a nucleic acid molecule, refers to an expression pattern in a non-naturally occurring plant that is distinct from the expression pattern in a comparable naturally occurring plant. Thus, one skilled in the art understands that ectopic expression of a nucleic acid molecule encoding an NHX-related gene product can refer to expression in a cell type other than a cell type in which the nucleic acid molecule normally is expressed, or at a time other than a time at which the nucleic acid molecule normally is expressed, or at a level other than the level at which the nucleic acid molecule normally is expressed. For example, under control of a constitutive promoter such as the cauliflower mosaic virus 35S promoter, NHX-1 is expressed in the leaves, thus, is ectopically expressed.

The term “non-halophyte,” as used herein means a plant that is not naturally morphologically and/or physiologically adapted to grow in salt rich soils or salt laden air. A non-halophyte is a plant variety that has a relative yield decrease of 50% or more at 200 mM NaCl (the equivalent of about 20 dS/m) when compared to the plant variety grown at optimal salinity levels which are below 200 mM NaCl. The invention is suitable for even more salt sensitive plant varieties which have a relative yield decrease of 50% or more at 180 mM NaCl, 160 mM NaCl, 140 mM NaCl, 120 mM NaCl, 100 mM NaCl or 80 mM NaCl. Table IV lists the relative yield decrease for various non-halophyte crop plants.

The term “saline-intolerant plants” as used herein means a plant variety that cannot complete its life cycle in growth media containing a salinity level above 200 mM NaCl. The invention is suitable for even more highly saline-intolerant plant varieties that cannot complete their life cycle in growth media containing a salinity level above 180 mM NaCl, 160 mM NaCl, 140 mM NaCl, 120 mM NaCl, 100 mM NaCl and even 7 mM NaCl.

Increased Potassium Levels

The term “increased potassium levels,” as used herein in reference to a fruit or vegetable produced by a non-naturally occurring berry plant or bush, fruit or vegetable producing plant varieties of the invention, means higher potassium levels when grown at elevated salt conditions as compared to the potassium levels of fruit or vegetables produced by a corresponding plant variety lacking a genetic modification introduced by human intervention such as an ectopically expressed nucleic acid molecule encoding an NHX related gene product such as a wild type plant. As disclosed herein in the Example, the seeds from a transgenic tomato plant ectopically expressing NHX-1 produce fruit that have potassium levels exhibiting almost 120% of the potassium levels of fruit produced from wild type tomato plants when grown under 200 mM NaCl.

It is recognized that there can be natural variation in the potassium levels of fruit or vegetables produced by a particular plant species or variety. However, fruit of increased potassium levels produced by a plant using a method of the invention readily can be identified by sampling a population of the produced fruit or vegetables and determining that the normal potassium distribution of fruit or vegetable is greater, on average, than the normal distribution of fruit or vegetables produced by the corresponding plant variety or species lacking a genetic modification introduced by human intervention such as an ectopically expressed nucleic acid molecule encoding an NHX related gene product. Thus, production of non-naturally occurring plants of the invention provides a means to skew the normal distribution of fruit or vegetable potassium levels produced by a plant, such that the fruit or vegetable potassium levels are, on average, at least about 5% greater, 10% greater, 15% greater, 20% greater, 25% greater, 30% greater, 50% greater, 75% greater, 100% greater, 200% greater, 300% greater, 400% greater or 500% greater than in the corresponding plant species that does not contain a genetic modification introduced by human intervention such as an ectopically expressed nucleic acid molecule encoding an NHX related gene product.

As used herein, the term “NHX-related gene product” means a gene product that has the same or similar function as At NHX-1 such that, when ectopically expressed in a plant, normal development is altered such that fruit or vegetables of increased potassium levels are produced. Arabidopsis NHX-1 is an example of an NHX related gene product as defined herein.

An NHX related gene product generally is characterized, in part, as containing a putative cation binding domain and an amiloride binding domain. An NHX-1 related gene product also generally is characterized by having an amino acid sequence that has at least about 40% amino acid identity with the amino acid sequence of Arabidopsis NHX-1. An NHX related gene product can have, for example, an amino acid sequence with greater than about 45% amino acid sequence identity with Arabidopsis NHX-1, preferably greater than about 50% amino acid identity with Arabidopsis NHX-1, preferably greater than about 55% amino acid sequence identity with Arabidopsis NHX-1, preferably greater than about 60% amino acid identity with Arabidopsis NHX-1, preferably greater than about 65% amino acid sequence identity with Arabidopsis NHX-1, preferably greater than about 75% amino acid identity with Arabidopsis NHX-1, more preferably greater than about 85% amino acid identity with Arabidopsis NHX-1, and can be a sequence having greater than about 90%, 95% or 97% amino acid identity with Arabidopsis NHX-1.

Preferably, an NHX-related gene product is orthologous to the plant species in which it is ectopically expressed. A nucleic acid molecule encoding tomato NHX, for example, can be ectopically expressed in a tomato plant to produce a non-naturally occurring tomato variety characterized by producing tomatoes with increased potassium levels. Similarly, a nucleic acid molecule encoding fruit tree NHX, for example, can be ectopically expressed in a fruit tree to produce a non-naturally occurring fruit tree characterized by producing fruit with increased potassium levels.

A nucleic acid molecule encoding an NHX-related gene product also can be ectopically expressed in a heterologous plant to produce a non-naturally occurring plant characterized by producing fruit with elevated potassium levels. NHX proteins have been cloned from a number of plant species (including Arabidopsis, tomato, sugar beets, petunia, rice, etc). indicating that they are widely conserved throughout the plant species. NHX-related gene products such as NHX orthologs also can be conserved and can function across species boundaries to produce fruit with increased potassium levels. Thus, ectopic expression of a nucleic acid molecule encoding an NHX-related gene product in a heterologous plant can alter fruit potassium levels. Furthermore, a nucleic acid molecule encoding an NHX-related gene product, for example, can be ectopically expressed in more distantly related heterologous plants, including dicotyledonous and monocotyledonous angiosperms and gymnosperms, fruit trees, berry plants and vines and, upon ectopic expression, can alter fruit potassium levels.

As used herein, the term “NHX-related gene product” encompasses an active segment of an NHX-related gene product, which is a polypeptide portion of an NHX-related gene product that, when ectopically expressed, increases fruit potassium levels. An active segment can be, for example, an amino terminal, internal or carboxy terminal fragment of Arabidopsis NHX-1 that, when ectopically expressed in a plant, produces fruit with elevated potassium levels. The skilled artisan will recognize that a nucleic acid molecule encoding an active segment of an NHX-related gene product can be used to generate a plant of the invention characterized by producing fruit with elevated potassium levels and in the related methods and kits of the invention described further below.

An active segment of an NHX-related gene product can be identified using the methods described in the Example or using other routine methodology. Briefly, a seed plant such as tomato can be transformed with a nucleic acid molecule under control of a constitutive regulatory element such as a tandem CaMV 35S promoter. Biochemical analysis of the plant reveals whether a seed plant ectopically expressing a particular polypeptide portion produces fruit with elevated potassium levels. For analysis of a large number of polypeptide portions of an NHX-related gene product, nucleic acid molecules encoding the polypeptide portions can be assayed in pools, and active pools subsequently subdivided to identify the active nucleic acid molecule.

In one embodiment, the invention provides a non-naturally occurring seed plant that is characterized by producing fruit with elevated potassium levels due to ectopic expression of a nucleic acid molecule encoding an NHX-related gene product having substantially the amino acid sequence of an NHX ortholog. As used herein, the term “NHX ortholog” means an ortholog of Arabidopsis NHX-1 and refers to an NHX-related gene product that, in a particular plant variety, has the highest percentage homology at the amino acid level to Arabidopsis NHX-1. An NHX-1 ortholog can be, for example the NHX-1 orthologs described in Table 2. Novel NHX ortholog cDNAs can be isolated from additional plant species using a nucleotide sequence as a probe and methods well known in the art of molecular biology (Glick and Thompson (eds.), Methods in Plant Molecular Biology and Biotechnology, Boca Raton, Fla.: CRC Press (1993); Sambrook et al. (eds.), Molecular Cloning: A Laboratory Manual (Second Edition), Plainview, N.Y.: Cold Spring Harbor Laboratory Press (1989), each of which is incorporated herein by reference).

As used herein, the term “substantially the amino acid sequence,” when used in reference to an NHX ortholog, is intended to mean a polypeptide or polypeptide segment having an identical amino acid sequence, or a polypeptide or polypeptide segment having a similar, non-identical sequence that is considered by those skilled in the art to be a functionally equivalent amino acid sequence. For example, an NHX-related gene product having substantially the amino acid sequence of Arabidopsis NHX-1 can have an amino acid sequence identical to the sequence of Arabidopsis NHX-1, or a similar, non-identical sequence that is functionally equivalent. In particular, a gene product that has “substantially the amino acid sequence” of an NHX ortholog can have one or more modifications such as amino acid additions, deletions or substitutions, including conservative or non-conservation substitutions, relative to the NHX-1 amino acid sequence, for example, provided that the modified polypeptide retains substantially the ability to increase fruit potassium levels when the nucleic acid molecule is ectopically expressed in the plant. Comparison of sequences for substantial similarity can be performed between two sequences of any length and usually is performed with sequences between about 6 and 1200 residues, preferably between about 10 and 100 residues and more preferably between about 25 and 35 residues. Such comparisons for substantial similarity are performed using methodology routine in the art.

The preferred percentage of sequence similarity for sequences of NHX orthologs includes nucleotide sequences having at least about: 48% similarity to SEQ ID NO:1. The similarity may also be at least about: 60% similarity, 75% similarity, 80% similarity, 90% similarity, 95% similarity, 97% similarity, 98% similarity, 99% similarity, or more preferably at least about 99.5% similarity, wherein the polypeptide has Na+/H+ transporter activity. The invention also includes salt tolerant plants made by transgenic expression of nucleic acid molecules encoding polypeptides, with the polypeptides having at least about: at least about: 48% similarity to SEQ ID NO:2. The similarity may also be at least about: 60% similarity, 75% similarity, 80% similarity, 90% similarity, 95% similarity, 97% similarity, 98% similarity, 99% similarity, or more preferably at least about 99.5% similarity, wherein the polypeptide Na+/H+ has transporter activity, to SEQ ID NO:2 (or a partial sequence thereof) considering conservative amino acid changes, wherein the polypeptide has Na+/H+ transporter activity. Sequence similarity is preferably calculated as the number of similar amino acids in a pairwise alignment expressed as a percentage of the shorter of the two sequences in the alignment. The pairwise alignment is preferably constructed using the Clustal W program, using the following parameter settings: fixed gap penalty=10, floating gap penalty=10, protein weight matrix=BLOSUM62. Similar amino acids in a pairwise alignment are those pairs of amino acids which have positive alignment scores defined in the preferred protein weight matrix (BLOSUM62). The protein weight matrix BLOSUM62 is considered appropriate for the comparisons described here by those skilled in the art of bioinformatics. (The reference for the clustal w program (algorithm) is Thompson, J. D., Higgins, D. G. and Gibson, T. J. (1994) CLUSTAL W: improving the sensitivity of progressive multiple sequence alignment through sequence weighting, positions-specific gap penalties and weight matrix choice. Nucleic Acids Research, 22:4673-4680; and the reference for BLOSUM62 scoring matrix is Henikoff, S. and Henikoff, J. G. (1993) Performance evaluation of amino acid substitution matrices. Proteins, 7:49-61.)

It is understood that minor modifications of primary amino acid sequence can result in an NHX-related gene product that has substantially equivalent or enhanced function as compared to the NHX ortholog from which it was derived. Further, various molecules can be attached to an NHX ortholog or active segment thereof, for example, other polypeptides, antigenic or other peptide tags, carbohydrates, lipids, or chemical moieties. Such modifications are included within the term NHX ortholog as defined herein.

One or more point mutations can be introduced into a nucleic acid molecule encoding an NHX ortholog to yield a modified nucleic acid molecule using, for example, site-directed mutagenesis (see Wu (Ed.), Meth. In Enzymol. Vol. 217, San Diego: Academic Press (1993); Higuchi, “Recombinant PCR” in Innis et al. (Ed.), PCR Protocols, San Diego: Academic Press, Inc. (1990), each of which is incorporated herein by reference). Such mutagenesis can be used to introduce a specific, desired amino acid insertion, deletion or substitution; alternatively, a nucleic acid sequence can be synthesized having random nucleotides at one or more predetermined positions to generate random amino acid substitutions. Scanning mutagenesis also can be useful in generating a modified nucleic acid molecule encoding substantially the amino acid sequence of an NHX ortholog.

Modified nucleic acid molecules can be routinely assayed for the ability to alter normal plant development such that fruit with elevated potassium levels are produced. For example, a nucleic acid molecule encoding substantially the amino acid sequence of an NHX ortholog can be ectopically expressed, for example, using a constitutive regulatory element such as the CaMV 35S promoter or using a tissue-specific regulatory element such as a fruit-selective regulatory element as described further below. If such ectopic expression results in a plant in which fruit or vegetables of elevated potassium levels are produced, the modified polypeptide or segment is an “NHX ortholog” as defined herein.

Other functional equivalent forms of the NHX-related gene product encoding nucleic acids can be identified using conventional DNA-DNA or DNA-RNA hybridization techniques. These nucleic acid molecules and the AtNHX sequences can be modified without significantly affecting their activity.

The plants of the present invention may therefore also be made by generating transgenic plants containing nucleic acid molecules that hybridize to one SEQ ID NO:1 or their complementary sequences, and that encode expression for peptides or polypeptides exhibiting substantially equivalent activity as that of an AtNHX polypeptide produced by SEQ ID NO:1 or their variants. Such nucleic acid molecules preferably hybridize to the sequences under low, moderate (intermediate), or high stringency conditions. (see Sambrook et al. (Most recent edition) Molecular Cloning: A Laboratory Manual, Cold Spring Harbor Laboratory Press, Cold Spring Harbor, N.Y.).

As used herein, the phrase “low stringency hybridization conditions” refers the following conditions and equivalents thereto: hybridization at 5×SSC, 2% SDS, and 100 μg/ml single stranded DNA at 40° C. for 8 hours, followed by at least one wash in 2×SSC, 0.2% SDS, at 40° C. for thirty minutes.

As used herein, the phrase “moderate stringency hybridization conditions” refers the following conditions and equivalents thereto: hybridization at 5×SSC, 2% SDS, and 100 μg/ml single stranded DNA at 50° C. for 8 hours, followed by at least one wash in 0.1×SSC, 0.1% SDS, at 50° C. for thirty minutes.

As used herein, the phrase “high stringency hybridization conditions” refers the following conditions and equivalents thereto: hybridization at 5×SSC, 2% SDS, and 100 μg/ml single stranded DNA at 65° C. for 8 hours, followed by at least one wash in 0.1×SSC, 0.1% SDS, at 65° C. for thirty minutes.

A non-naturally occurring plant of the invention that is characterized by producing fruit with elevated potassium levels can be one of a variety of plant species, including a monocotyledonous or dicotyledonous angiosperm or a gymnosperm.

The invention also provides a transgenic plant that is characterized by producing fruit with elevated potassium levels. A preferred method of making such a transgenic plant is by ectopic expression of an exogenous nucleic acid molecule encoding an NHX-related gene product. In a transgenic plant of the invention, the ectopically expressed exogenous nucleic acid molecule encoding an NHX-related gene product can be operatively linked to an exogenous regulatory element. In one embodiment, the invention provides a transgenic plant characterized by producing fruit with elevated potassium levels having an ectopically expressed exogenous nucleic acid molecule encoding an NHX-related gene product that is operatively linked to a constitutive regulatory element. The invention provides, for example, a transgenic plant that is characterized by producing fruit with elevated potassium levels due to ectopic expression of an exogenous nucleic acid molecule encoding substantially the amino acid sequence of an NHX ortholog operatively linked to a cauliflower mosaic virus 35S promoter.

In another embodiment, an exogenous constitutive or inducible regulatory element may be introduced to the plant such that the exogenous regulatory element is operably linked to an endogenous gene and alters the expression pattern of the gene in a manner that elevates the potassium level in the fruit. One example of this would be to transfect a plant with the cauliflower mosaic virus 35S promoter such that the promoter integrates in a way that it is operably linked to one of the plant's endogenous NHX-related genes.

In yet another embodiment, an exogenous NHX-related gene may be introduced to the plant such that the exogenous NHX-related gene is operably linked to an endogenous regulatory element which directs the expression of the gene in a manner that elevates the potassium level in the fruit.

Yet another embodiment is to transfect a plant with an NHX-related gene with out a promoter in such a way that it integrates operably linked to an endogenous promoter in the plant. One example of this would be to transfect a plant with the atNHX1 gene such that the gene integrates in a way that it is operably linked to one of the plant's endogenous strong promoters.

As used herein, the term “transgenic” refers to a seed plant that contains an exogenous nucleic acid molecule, which can be derived from the same plant species or from a heterologous plant species.

The term “exogenous,” as used herein in reference to a nucleic acid molecule and a transgenic plant, means a nucleic acid molecule originating from outside the plant. An exogenous nucleic acid molecule can have a naturally occurring or non-naturally occurring nucleotide sequence. One skilled in the art understands that an exogenous nucleic acid molecule can be a heterologous nucleic acid molecule derived from a different plant species than the plant into which the nucleic acid molecule is introduced or can be a nucleic acid molecule derived from the same plant species as the seed plant into which it is introduced.

The term “operatively linked,” as used in reference to a regulatory element and a nucleic acid molecule, such as a nucleic acid molecule encoding an NHX-related gene product, means that the regulatory element confers regulated expression upon the operatively linked nucleic acid molecule. Thus, the term “operatively linked,” as used in reference to an exogenous regulatory element such as a constitutive regulatory element and a nucleic acid molecule encoding an NHX-related gene product, means that the constitutive regulatory element is linked to the nucleic acid molecule encoding an NHX-related gene product such that the expression pattern of the constitutive regulatory element is conferred upon the nucleic acid molecule encoding the NHX-related gene product. It is recognized that a regulatory element and a nucleic acid molecule that are operatively linked have, at a minimum, all elements essential for transcription, including, for example, a TATA box.

Constitutive Regulatory Elements

As used herein, the term “constitutive regulatory element” means a regulatory element that confers a level of expression upon an operatively linked nucleic molecule that is relatively independent of the cell or tissue type in which the constitutive regulatory element is expressed. A constitutive regulatory element that is expressed in a plant generally is widely expressed in a large number of cell and tissue types.

A variety of constitutive regulatory elements useful for ectopic expression in a transgenic plant of the invention are well known in the art. The cauliflower mosaic virus 35S (CaMV 35S) promoter, for example, is a well-characterized constitutive regulatory element that produces a high level of expression in all plant tissues (Odell et al., Nature 313:810-812 (1985)). The CaMV 35S promoter can be particularly useful due to its activity in numerous diverse plant species (Benfey and Chua, Science 250:959-966 (1990); Futterer et al., Physiol. Plant 79:154 (1990); Odell et al., supra, 1985). A tandem 35S promoter, in which the intrinsic promoter element has been duplicated, confers higher expression levels in comparison to the unmodified 35S promoter (Kay et al., Science 236:1299 (1987)). Other constitutive regulatory elements useful for ectopically expressing a nucleic acid molecule encoding an NHX-related gene product in a transgenic seed plant of the invention include, for example, the cauliflower mosaic virus 19S promoter; the Figwort mosaic virus promoter; and the nopaline synthase (nos) gene promoter (Singer et al., Plant Mol. Biol. 14:433 (1990); An, Plant Physiol. 81:86 (1986)).

Additional constitutive regulatory elements including those for efficient ectopic expression in monocots also are known in the art, for example, the pEmu promoter and promoters based on the rice Actin-1 5′ region (Last et al., Theor. Appl. Genet. 81:581 (1991); Mcelroy et al., Mol. Gen. Genet. 231:150 (1991); Mcelroy et al., Plant Cell 2:163 (1990)). Chimeric regulatory elements, which combine elements from different genes, also can be useful for ectopically expressing a nucleic acid molecule encoding an NHX-related gene product (Comai et al., Plant Mol. Biol. 15:373 (1990)). One skilled in the art understands that a particular constitutive regulatory element is chosen based, in part, on the plant species in which a nucleic acid molecule encoding an NHX-related gene product is to be ectopically expressed and on the desired level of expression.

An exogenous regulatory element useful in a transgenic plant of the invention also can be an inducible regulatory element, which is a regulatory element that confers conditional expression upon an operatively linked nucleic acid molecule, where expression of the operatively linked nucleic acid molecule is increased in the presence of a particular inducing agent or stimulus as compared to expression of the nucleic acid molecule in the absence of the inducing agent or stimulus. Particularly useful inducible regulatory elements include copper-inducible regulatory elements (Mett et al., Proc. Natl. Acad. Sci. USA 90:4567-4571 (1993); Furst et al., Cell 55:705-717 (1988)); tetracycline and chlor-tetracycline-inducible regulatory elements (Gatz et al., Plant J. 2:397-404 (1992); Roder et al., Mol. Gen. Genet. 243:32-38 (1994); Gatz, Meth. Cell Biol. 50:411-424 (1995)); ecdysone inducible regulatory elements (Christopherson et al., Proc. Natl. Acad. Sci. USA 89:6314-6318 (1992); Kreutzweiser et al., Ecotoxicol. Environ. Safety 28:14-24 (1994)); heat shock inducible regulatory elements (Takahashi et al., Plant Physiol. 99:383-390 (1992); Yabe et al., Plant Cell Physiol. 35:1207-1219 (1994); Ueda et al., Mol. Gen. Genet. 250:533-539 (1996)); and lac operon elements, which are used in combination with a constitutively expressed lac repressor to confer, for example, IPTG-inducible expression (Wilde et al., EMBO J. 11:1251-1259 (1992)).

An inducible regulatory element useful in the transgenic seed plants of the invention also can be, for example, a nitrate-inducible promoter derived from the spinach nitrite reductase gene (Back et al., Plant Mol. Biol. 17:9 (1991)) or a light-inducible promoter, such as that associated with the small subunit of RuBP carboxylase or the LHCP gene families (Feinbaum et al., Mol. Gen. Genet. 226:449 (1991); Lam and Chua, Science 248:471 (1990)). Additional inducible regulatory elements include salicylic acid inducible regulatory elements (Uknes et al., Plant Cell 5:159-169 (1993); Bi et al., Plant J. 8:235-245 (1995)); plant hormone-inducible regulatory elements (Yamaguchi-Shinozaki et al., Plant Mol. Biol. 15:905 (1990); Kares et al., Plant Mol. Biol. 15:225 (1990)); and human hormone-inducible regulatory elements such as the human glucocorticoid response element (Schena et al., Proc. Natl. Acad. Sci. USA 88:10421 (1991)).

It should be recognized that a non-naturally occurring plant of the invention, which contains an ectopically expressed nucleic acid molecule encoding an NHX-related gene product, also can contain one or more additional modifications, including naturally and non-naturally occurring mutations that can, for example, increase fruit potassium levels.

The invention further provides a method of producing a non-naturally occurring plant characterized by producing fruit with elevated potassium levels. One method is practiced by ectopically expressing a nucleic acid molecule encoding an NHX-related gene product in the plant, whereby fruit potassium levels are increased due to ectopic expression of the nucleic acid molecule. In one embodiment, the method is practiced by introducing an exogenous nucleic acid molecule encoding an NHX-related gene product into the plant.

As discussed above, the term “ectopically” refers to expression of a nucleic acid molecule encoding an NHX-related gene product in a cell type other than a cell type in which the nucleic acid molecule is normally expressed, at a time other than a time at which the nucleic acid molecule is normally expressed or at an expression level other than the level at which the nucleic acid molecule normally is expressed.

Actual ectopic expression of an NHX-related gene product is dependent on various factors. The ectopic expression can be widespread expression throughout most or all plant tissues or can be expression restricted to a small number of plant tissues, and can be achieved by a variety of routine techniques. Mutagenesis, including seed or pollen mutagenesis, can be used to generate a non-naturally occurring seed plant, in which a nucleic acid molecule encoding an NHX-related gene product is ectopically expressed. Ethylmethane sulfonate (EMS) mutagenesis, transposon mediated mutagenesis or T-DNA mediated mutagenesis also can be useful in ectopically expressing an NHX-related gene product to produce a seed plant that produces seeds of increased size (see, generally, Glick and Thompson, supra, 1993). While not wishing to be bound by any particular mechanism, ectopic expression in a mutagenized plant can result from inactivation of one or more negative regulators of NHX, for example.

Ectopic expression of an NHX-related gene product also can be achieved by expression of a nucleic acid molecule encoding an NHX-related gene product from a heterologous regulatory element or from a modified variant of its own promoter. Heterologous regulatory elements include constitutive regulatory elements, which result in expression of the NHX-related gene product in the fruit as well as in a variety of other cell types, and seed-selective regulatory elements, which produce selective expression of an NHX-related gene product in a limited number of plant tissues, including one or more fruit tissues.

Ectopic expression of a nucleic acid molecule encoding an NHX-related gene product can be achieved using an endogenous or exogenous nucleic acid molecule encoding an NHX-related gene product. A recombinant exogenous nucleic acid molecule can contain a heterologous regulatory element that is operatively linked to a nucleic acid sequence encoding an NHX-related gene product. Methods for producing the desired recombinant nucleic acid molecule under control of a heterologous regulatory element and for producing a non-naturally occurring plant of the invention are well known in the art (see, generally, Sambrook et al., supra, 1989; Glick and Thompson, supra, 1993).

Transformation

An exogenous nucleic acid molecule can be introduced into a plant for ectopic expression using a variety of transformation methodologies including Agrobacterium-mediated transformation and direct gene transfer methods such as electroporation and microprojectile-mediated transformation (see, generally, Wang et al. (eds), Transformation of Plants and Soil Microorganisms, Cambridge, UK: University Press (1995), which is incorporated herein by reference). Transformation methods based upon the soil bacterium Agrobacterium tumefaciens are particularly useful for introducing an exogenous nucleic acid molecule into a seed plant. The wild type form of Agrobacterium contains a Ti (tumor-inducing) plasmid that directs production of tumorigenic crown gall growth on host plants. Transfer of the tumor-inducing T-DNA region of the Ti plasmid to a plant genome requires the Ti plasmid-encoded virulence genes as well as T-DNA borders, which are a set of direct DNA repeats that delineate the region to be transferred. An Agrobacterium-based vector is a modified form of a Ti plasmid, in which the tumor inducing functions are replaced by the nucleic acid sequence of interest to be introduced into the plant host.

Agrobacterium-mediated transformation generally employs cointegrate vectors or, preferably, binary vector systems, in which the components of the Ti plasmid are divided between a helper vector, which resides permanently in the Agrobacterium host and carries the virulence genes, and a shuttle vector, which contains the gene of interest bounded by T-DNA sequences. A variety of binary vectors are well known in the art and are commercially available, for example, from Clontech (Palo Alto, Calif.). Methods of coculturing Agrobacterium with cultured plant cells or wounded tissue such as leaf tissue, root explants, hypocotyledons, stem pieces or tubers, for example, also are well known in the art (Glick and Thompson, supra, 1993). Wounded cells within the plant tissue that have been infected by Agrobacterium can develop organs de novo when cultured under the appropriate conditions; the resulting transgenic shoots eventually give rise to transgenic plants that ectopically express a nucleic acid molecule encoding an NHX-related gene product. Agrobacterium also can be used for transformation of whole seed plants as described in Bechtold et al., C.R. Acad. Sci. Paris. Life Sci. 316:1194-1199 (1993), (which is incorporated herein by reference). Agrobacterium-mediated transformation is useful for producing a variety of transgenic seed plants (Wang et al., supra, 1995) including transgenic plants of the Brassicaceae family, such as rapeseed and flax, and transgenic plants of the Fabaceae family such as soybean, pea, lentil and bean.

Microprojectile-mediated transformation also can be used to produce a transgenic seed plant that ectopically expresses an NHX-related gene product. This method, first described by Klein et al. (Nature 327:70-73 (1987), which is incorporated herein by reference), relies on microprojectiles such as gold or tungsten that are coated with the desired nucleic acid molecule by precipitation with calcium chloride, spermidine or PEG. The microprojectile particles are accelerated at high speed into an angiosperm tissue using a device such as the BIOLISTIC PD-1000 (Biorad; Hercules Calif.).

Microprojectile-mediated delivery or “particle bombardment” is especially useful to transform seed plants that are difficult to transform or regenerate using other methods. Microprojectile-mediated transformation has been used, for example, to generate a variety of transgenic plant species, including cotton, tobacco, corn, hybrid poplar and papaya (see Glick and Thompson, supra, 1993) as well as cereal crops such as wheat, oat, barley, sorghum and rice (Duan et al., Nature Biotech. 14:494-498 (1996); Shimamoto, Curr. Opin. Biotech. 5:158-162 (1994), each of which is incorporated herein by reference). In view of the above, the skilled artisan will recognize that Agrobacterium-mediated or microprojectile-mediated transformation, as disclosed herein, or other methods known in the art can be used to produce a transgenic seed plant of the invention.

Kits

Kits for generating a transgenic plant characterized by producing fruit of elevated potassium levels are provided herein. The kits of the invention include a nucleic acid molecule encoding an NHX-related gene product and a regulatory element. In a kit of the invention, the NHX-related gene product can have, for example, substantially the amino acid sequence of an NHX ortholog. If desired, a kit for generating a transgenic plant characterized by producing fruit of elevated potassium levels can include a plant expression vector containing a nucleic acid molecule encoding an NHX-related gene product operatively linked to a seed-selective regulatory element.

Nucleic acid molecules encoding NHX-related gene products, such as those having substantially the amino acid sequence of an NHX ortholog, have been described hereinabove. A kit of the invention can contain one of a variety of nucleic acid molecules encoding NHX-related gene products and any regulatory element, such as an element described hereinabove.

If desired, a kit of the invention also can contain a plant expression vector. As used herein, the term “plant expression vector” means a self-replicating nucleic acid molecule that provides a means to transfer an exogenous nucleic acid molecule into a seed plant host cell and to express the molecule therein. Plant expression vectors encompass vectors suitable for Agrobacterium-mediated transformation, including binary and cointegrating vectors, as well as vectors for physical transformation.

Plant expression vectors can be used for transient expression of the exogenous nucleic acid molecule, or can integrate and stably express the exogenous sequence. One skilled in the art understands that a plant expression vector can contain all the functions needed for transfer and expression of an exogenous nucleic acid molecule; alternatively, one or more functions can be supplied in trans as in a binary vector system for Agrobacterium-mediated transformation.

In addition to containing a nucleic acid molecule encoding an NHX-related gene product operatively linked to a seed-selective regulatory element, a plant expression vector of the invention can contain, if desired, additional elements. A binary vector for Agrobacterium-mediated transformation contains one or both T-DNA border repeats and can also contain, for example, one or more of the following: a broad host range replicon, an ori T for efficient transfer from E. coli to Agrobacterium, a bacterial selectable marker such as ampicillin and a polylinker containing multiple cloning sites.

A plant expression vector for physical transformation can have, if desired, a plant selectable marker and can be based on a vector such as pBR322, pUC, pGEM and M13, which are commercially available, for example, from Pharmacia (Piscataway, N.J.) or Promega (Madison, Wis.). In plant expression vectors for physical transformation of a seed plant, the T-DNA borders or the ori T region can optionally be included but provide no advantage.

The invention also provides a method of generating a non-naturally occurring plant that is characterized by producing fruit or vegetables of increased potassium levels. The method includes the step of ectopically expressing a nucleic acid molecule encoding an NHX-family gene product in the plant, whereby fruit potassium levels are increased due to ectopic expression of the nucleic acid molecule. In one embodiment, the method includes the step of introducing an exogenous nucleic acid molecule encoding an NHX-family gene product into the plant.

Examples of a non-naturally occurring seed plant of the invention characterized by producing fruit of increased potassium levels include vegetables such as tomatoes, citrus trees, such as orange trees, grapefruit trees, lemon trees and lime trees. A non-naturally occurring plant of the invention characterized by producing fruit of increased potassium level also can be a plant that bears, for example, grapes, apples, pears, peaches, plums, cherries, bananas, blackberries, blueberries, raspberries, strawberries, pineapples, dates, avocados, olives, tomatoes, cucumbers or eggplants, such fruits having an increased potassium level as compared to the fruit produced by the corresponding wild type plant.

The invention will be better understood by reference to the following non-limiting example.

### EXAMPLE

Experimental Protocol

Plant Material and Transgenic Plants

Lycopersicon esculentum (cv Moneymaker) seeds were germinated on Murashige and Skoog medium (MS). Cotyledon explants were excised from 7 day-old seedlings, cut in half and cultured overnight on a one day-old feeder layer consisting of 3 ml of a 7 day-old sugar beet suspension culture plated and overlaid with a sterile Whatman filter paper. The binary Ti vector pBI121 was used for transformation. The GUS gene26 of the binary vector was replaced with the AtNHX1 gene to gain the new expression construct pHZX1. pHZX1 was electroporated into Agrobacterium tumefaciens strain LBA4404. For co-cultivation, 1 ml of pHZX1 containing Agrobacterium were inoculated into 15 ml LB medium containing 50 mg/l kanamycin, 50 mg/l rifampicin and 200 μM acetone-syringone. After two days of co-cultivation with Agrobacterium, the explants were transferred to selective regeneration medium 27. Regenerated shoots were transferred to fresh medium bi-weekly. When the green shoots were 1-2 cm tall, they were separated from the calli and transferred onto rooting medium containing modified MS salts27. About 98% shoots can form roots in two weeks. Rooted shoots were transplanted to soil and plants regenerated. T1 seeds were grown on plates containing MS medium and 100 mg/l kanamycin and homozygous seeds selected.

For salt tolerance experiments, wild type and two independent lines (T2) of transgenic plants were grown hydroponically. Seeds were germinated in agar plates containing MS medium under continuous light at 25 ° C. Two weeks after germination, sixty of each wild-type and transgenic seedlings were transferred to six hydroponic tanks, containing 20 seedlings each tank, and grown in the greenhouse. Day temperature was maintained at 26±2° C. and night temperature was 22±2° C. Relative humidity was maintained at 50±10%. Plants were grown under a 14 h/10 h light/dark photoperiod. Supplemental lighting consisted of eight high-pressure sodium lamps, and resulted in a total (sunlight and supplemental light) of approximately 1,250 μmol/m2 s. The nutrient solution was obtained by mixing 1.2 g per liter of stock fertilizer (tomato fertilizer, Plant-Prod, Brampton, Ontario) and 1 g per liter of CaNO3. The final nutrient solution contained (in mg/l) 200 N, 54 P, 256 K, 147 Ca, 42 Mg, micronutrients and was supplemented with 5 mM or 200 mM NaCl. The nutrient solution was replaced every 6 days and the roots were kept under constant aeration.

Membrane Isolation and Western Blots

Membrane fractions were isolated from shoots of 4-week-old plants or tomato fruits from mature plants as described 5. Western blots of the different membrane fractions were performed as described4.

Transport Assays

The cation/H+ exchange activity was measured by following the pH dependent fluorescence quenching of acridine orange5. An acidic-inside pH gradient across the tonoplast vesicles was obtained by activation of the vacuolar H+-PPiase. Twenty μg of tonoplast vesicles were added to 0.8 ml buffer containing 0.25 M Mannitol, 5 mM Tris/MES (pH 8.0), 2 mM dithiotreitol, 25 mM KCl, 0.8 mM Tris-PPi and 5 μM acridine orange. Proton translocation was initiated by the addition of 1 mM Mg2+ and the change in fluorescence was monitored as described5. When a steady-state pH gradient (acidic inside) was formed, PPi-dependent H+-transport activity was stopped by the addition of AMDP and the changes in rate of fluorescence recovery were determined in the presence and absence of 50 mM NaCl.

Leaf and Fruit Chemical Analysis

Chemical analysis from 3-month old plants was performed. Fully-expanded mature leaves from the six most lower basal nodes (old leaves), developing leaves from the six most upper apical nodes (young leaves), roots and fruits were collected and dried at 70° C. for 24 h and the material ground to a find powder. Tomatoes were collected at the mature green/red ripe stage and were allowed one week of further maturation at the bench at room temperature (22 ° C.) before analysis. For the determination of soluble sugars, 100 mg of each sample was resuspended in 2 ml of water, sonicated and centrifuged for 10 min at 2,500×g. Soluble sugar and proline contents were determined in the supernatant as described. Ion contents were determined by atomic absorption spectrophotometry and chloride content by titration. Water content was calculated as (FW-DW)/FW, where FW and DW are the fresh and dry weight, respectively. Dry weight was obtained by placing the material at 70° C. until a constant weight was obtained. For the determination of soluble solid contents, the tomatoes were strained through a 20 μm mesh and Brix readings of the juice were obtained by refractrometry. Brix readings (oBrix) represent the concentrations of soluble solids as a percentage of total fresh weight.

Results and Discussion

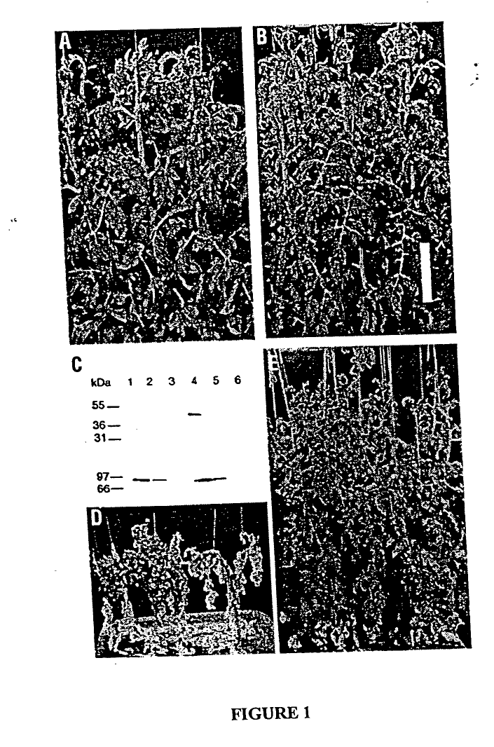

A construct containing the Arabidopsis thaliana AtNHX1, coding for a vacuolar Na+/H+ antiport, was introduced into the genome of Lycopersicon esculentum cv Moneymaker. Forty-seven transgenic plants were obtained and six homozygous lines from these transgenic plants were obtained in the T2 generation (data not shown). Two of these homozygous lines were used in our experiments. These two lines were chosen because they grew more vigorously in high salinity. The overexpression of the vacuolar Na+/H+ antiport did not affect the growth of the transgenic plants (only one line of transgenic plants is shown) since similar growth was observed when the wild-type and the transgenic plants were grown in the presence of 5 mM NaCl (FIG. 1A,B). Immunoblots of membrane fractions isolated from wild-type and transgenic plants only detected AtNHX1 in the tonoplast-enriched fractions from transgenic plants (FIG. 1C), indicating the proper targeting of the Na+/H+ antiport to the vacuoles. In order to assess whether the enhanced expression of the vacuolar Na+/H+ antiport would allow plants to grow in high salt conditions, wild-type and transgenic plants were grown in the presence of 200 mM NaCl, a concentration that inhibits the growth of almost all crop plants. The growth of the wild-type plants was severely affected by the presence of 200 mM NaCl in the growth solution, plant growth was inhibited, most of the plants died or were severely stunted (FIG. 1D). On the other hand, the transgenic plants grew, flowered and produced fruit (FIG. 1E).

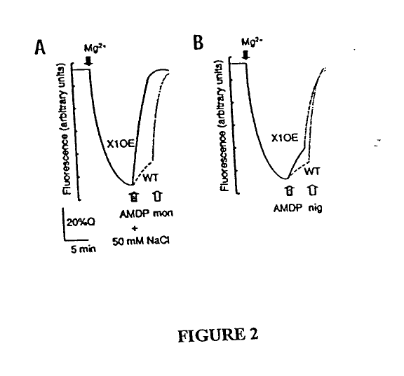

To confirm that the presence of the Na+/H+ antiport protein resulted in increased Na+/H+ exchange, we monitored H+-dependent Na+ movements in tonoplast vesicles isolated from leaves. The vesicular lumen was acidified by the activation of the vacuolar H+-PPIase in the presence of K+ ions, since the H+-PPIase activity is K+ dependent7. Once the pH gradient was established, the H+-pump activity was stopped by the addition of AMDP (amino-methylene-diphosphonate)8, NaCl was added and the rates of Na+/H+ exchange measured (FIG. 2A). Tonoplast vesicles isolated from transgenic plants displayed Na+/H+ exchange rates 7-fold higher than those from vesicles isolated from wild-type plants. Interestingly, K+/H+ exchange was also observed in the tonoplast vesicles after the addition of AMDP, in the absence of external Na+, (FIG. 2B) and the rates of K+/H+ exchange were significantly higher in vesicles isolated from the transgenic plants. These results indicate that the vacuolar Na+/H+ antiport was also able to mediate K+/H+ exchange, albeit with a lower specificity for K+than for Na+. K+ ions are involved in a wide number of physiological processes and vacuolar pools generate the turgor needed to drive cell expansion9. Under K+ deficient growth conditions, vacuolar K+ concentrations decline while the cytosolic K+ concentrations remain relatively constant10. Cytosolic K+ concentrations decline only when the vacuolar K+ concentrations decrease to values around 20 mM11. The decrease in cytosolic K+ concentrations with the concomitant increase in cytosolic Na+/K+ ratio is the basis of cytosolic Na+ toxicity12. Given the cytosol-negative electrical potential difference at the tonoplast, an active K+ translocation mechanism into the vacuole has to be considered. Evidence of a K+/H+ antiport was found in tonoplast-enriched fractions from different plants6. Although the Arabidopsis sequencing project is completed, only putative K+/H+ antiports with similarity to the glutathione-regulated potassium-efflux system of E. coli13 have been sequenced (Accession numbers AAF78418, AAD10158, CCAB80872). Although their putative function has not yet been characterized in plants, in bacteria and yeast these transporters function as plasma membrane-bound potassium exchangers13,14. Although the role of vacuolar Na+/H+ antiports in glycophytes has yet to be established, its ubiquity in plants (Blumwald, in preparation) and its ability to mediate K+ transport would suggest that the vacuolar Na+/H+ antiport could also play a role in cellular K+ homeostasis.

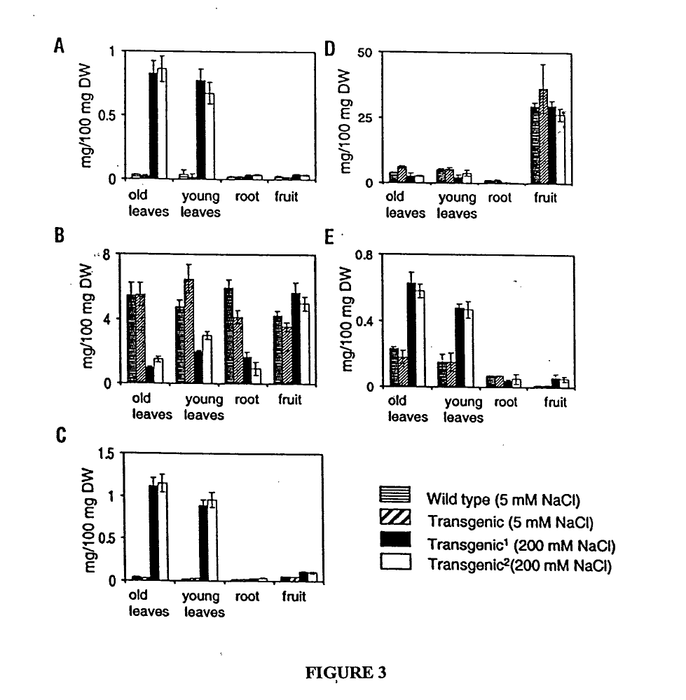

We determined the ion, sugar, and proline contents of wild-type and transgenic plants grown at low (5 mM) NaCl and two independent transgenic lines grown at high (200 mM) NaCl (FIG. 3). It should be noted that a comparison with wild-type plants grown at high salinity was not possible since all of the wild-type plants grown in these conditions were dead. At low salinity, no significant differences were seen in the content of Na+ (FIG. 3A), K+(FIG. 3B), Cl−(FIG. 3C) soluble sugars (FIG. 3D) or proline (FIG. 3D) of all tissues. Dramatic changes were seen in transgenic plants grown at high salinity. A 28- and 20-fold increase in Na+ content was seen in fully developed mature (old) and developing (young) leaves, respectively (FIG. 3A), and a similar increase in Cl− content was also observed (FIG. 3C). The K+ content of old leaves, young leaves and roots decreased a 5-, 2- and 4-fold, respectively (FIG. 3B). While no significant difference in soluble sugars was observed during growth in high salinity (FIG. 3D), a 3- and 5-fold increase in proline content was seen in leaves and fruits, respectively (FIG. 3E). The accumulation of proline in response to high salinity is well documented. Many prokaryotic and eukaryotic organisms accumulate proline during osmotic and salt stress15,16. Proline contributes to osmotic adjustment17, the protection of macromolecules during dehydration 18, and as a hydroxyl radical scavenger 19. Evidence supporting the role of proline during salt stress was obtained based on salt tolerance in transgenic tobacco plants with enhanced levels of proline biosynthesis20 and salt tolerance of Arabidopsis with suppressed levels of proline degradation21.

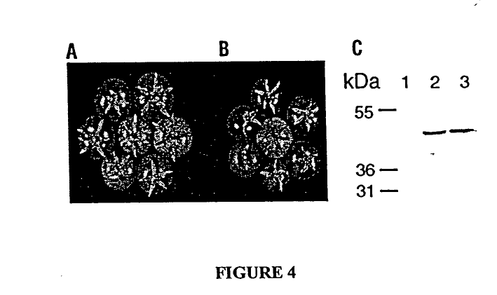

Taken together, our results demonstrate the ability of the transgenic plants to utilize salty water for growth. In spite of the high Na+ and Cl− content in the leaves of the transgenic plants grown at 200 mM NaCl, only a marginal increase in the Na+ and Cl− content of the fruits was observed. The K+ content of the leaves from transgenic plants grown in salt decreased while the K+ content of the transgenic fruits was higher than the K+ content of the fruits from plants grown at low salinity. These results clearly demonstrate that the enhanced accumulation of Na+ , mediated by the vacuolar Na+/H+ antiport, allowed the transgenic plants to ameliorate the toxic effects of Na+ and the transgenic plants overcame salt-induced impaired nutrient acquisition7. Notably, transgenic plants grown in the presence of 200 mM NaCl produced fruits (FIGS. 4A,B and Table 1). While the transgenic leaves accumulated Na+ to almost 1% of their dry weight, the fruits displayed only a marginal increase in Na+ content and a 25% increase in K+ content. The number of fruits per plant was similar, and although the fruits from the transgenic plants grown in 200 mM NaCl were somewhat smaller, no significant difference was observed in their water content or total soluble solids content (Table 1). The low Na+ content of the transgenic fruits cannot be due to the lack of vacuolar Na+/H+ antiport since the protein was present in the fruit tissue (FIG. 4C). It has been demonstrated that in expanding fruit of many plant species, including tomato, more than 90% of the water transported into the fruit occurs through the phloem22,23,24. Thus the ability to maintain a high cytosolic K+/Na+ concentration ratio along the symplastic pathway was most probably responsible for the low Na+ content of the fruits.

Worldwide, more than 60 million hectares of irrigated land (representing 25% of the total irrigated acreage in the world) have been damaged by salt25. Our findings suggests the feasibility of producing salt tolerant transgenic plants that will produce edible crops.

### REFERENCES

Yeo, A. R., Yeo, M. E., Flowers, S. A. Flowers, T. J. Screening of rice (Oryza sativa L.) genotypes for physiological characters contributing to salinity resistance, and their relationship to overall performance. Theor. Appl. Genet. 79, 377-384 (1988).
Cuartero, J., Fernandez-Munoz, R. Tomato and salinity. Sci. Hort. 78, 83-125 (1999).
Zhu, J-K. Genetic analysis of plant salt tolerance using Arabidopsis. Plant Physiol. 124, 941-948 (2000).
Apse, M. P., Aharon, G. S., Snedden, W. S. & Blumwald, E. Salt tolerance conferred by overexpression of a vacuolar Na+/H+ antiport in Arabidopsis. Science 285, 1256-1258 (1999).
Blumwald, E. & Poole, R. J. Na⁺/H⁺ antiport in isolated tonoplast vesicles from storage tissue of Beta vulgaris. Plant Physiol. 78, 163-167 (1985).
Blumwald, E. & Gelli, A. Secondary inorganic ion transport in plant vacuoles. Adv. Bot. Res. 25, 401-407 (1997).
Rea, P. A. & Sanders, D. Tonoplast energyzation: two H⁺-pumps, one membrane. Physiol. Plant. 71, 131-141 (1987).
Drozdowicz, Y. M. et al. A thermostable vacuolar-type membrane pyrophosphatase from the archeon Pyrobaculum aerophilum: implications for the origins of pyrophosphatase-energized pumps. FEBS Lett. 460, 505-512 (1999).
Marschner, H. Mineral nutrition of higher plants. (Academic Press, New York; 1995).
Walker, D J., Leigh, R A. & Miller, A J. Potassium homeostasis in vacuolate plant cells. Proc. Natl. Acad. Sci. USA 93, 10510-10514 (1996).
Leigh, R A. & Wynn Jones, R G. A hypothesis relating critical potassium concentrations for growth and distribution and functions of this ion in the plant cell. New Phytol. 97,1-13. (1984).
Maathuis, F J M. & Amtmann, A. K⁺ nutrition and Na⁺ toxicity: the basis of cellular K⁺/Na⁺ ratios. Ann. Bot. 84, 123-133. (1999).
Munro, A W., Ritchi, G Y., Lamb, R M., Douglas, R M., & Booth, I R. The cloning and DNA sequence of the gene for the glutathion-regulated potassium-efflux system KefC of Escherichia coli. Mol. Microbiol. 5, 607-616(1991).
Ramirez, J., Ramirez, O., Saldana, C., Coria, R., & Pena, Antonio. A Saccharomyces cerevisiae mutant lacking a K⁺/H⁺ exchanger. J Bacteriol. 180, 5860-5865 (1998).
Czonka, L. N. & Hanson, A. D. Prokaryotic osmoregulation: genetics and physiology. Annu Rev. Microbiol. 45, 569-606 (1991).
Schobert, B. Is there an osmotic regulatory mechanism in algae and higher plants? J Theor. Biol. 68, 17-26 (1977).
LeRudulier, D., Strom, A. R., Dandekar, A. M., Smith, L. T. & Valentine, R. C. Molecular biology of osmoregulation. Science 224, 1064-1068.
Yancey, P., Clark, M., Hand, S., Bowlus, R. & Somero, G. Living with water stress: evolution of osmolyte systems. Science 217, 1214-22 (1982).
Smirnoff, N. & Cumbes, Q. J. Hydroxyl radial scavenging activity of compatible solutes. Phytochemistry 28, 1057-1060 (1989).
Kishor, P. B. K, Hong, Z., Miao, G-H., Hu, C-A. A. & Verma, D P S. Overexpression of Δ¹-pyrroline-5-carboxylate synthetase increases proline production and confers osmotolerance in transgenic plants. Plant Physiol. 108, 1387-1394 (1995).
Nanjo, T. et al. Antisense supression of proline degradation improves tolerance to freezing and salinity in Arabidopsis thaliana. FEBS Lett. 461, 205-210 (1999).
Ehret, D. L. & Ho, L. C. Effects of osmotic potential in nutrient solution on diurnal growth of tomato fruit. J Exp. Bot. 37, 1294-1302 (1986).
Lee, D.R. A Vasculature of the abcission zone of tomato fruit: implications for transport. Can J Bot. 67, 1898-1902.
Davies, W. J., Bacon, M. A., Thompson, D. S., Sobeih, W. & Rodriguez, L. G. Regulation of leaf and fruit growth in plants growing in drying soil: exploitation of the plants' chemical signalling system and hydraulic architecture to increase the efficiency of water use in agriculture. J. Exp. Bot. 51, 1617-1626 (2000).
Ghassemi, F., Jakeman, A. & Nix, H. Salinization of land and water resources: Human causes, extent, Management and case studies. (University of South Wales Press, Sydney 1995).
Jefferson, R. A., Kavanagh, T. A. & Bevan, M. W. GUS fusions: beta-glucoronidase as a sensitive and versatile gene fusion marker in higher plants. EMBO J. 6, 3901-3907 (1987).
Thomas, B. R. & Pratt, D. Efficient hybridization between Lycopersicon esculentum and L. peruvianum via embryo callus. Theor. Appl. Genet. 59, 215-219 (1981).
Dubois, M., Gilles, R A., Hamilton, J K., Roberts, P A. & Smith, F. Colorimetric method for determination of sugar and related substances. Anal. Chem. 28, 350-356 (1956).

Bates, L. S., Waldren, R. P. & Teare, I. D. Rapid determination of proline for water-stress studies. Plant & Soil 39, 205-207 (1973).

TABLE I
Plant and fruit yield of wild-type (WT) tomato plants grown in the
presence of 5 mM NaCl and T2 transgenic plants overexpressing
AtNHX1 (OEX1) grown in the presence of 5 mM and 200 mM NaCl.
Plants were harvested 12 weeks after germination. Each value is the
Mean ± SD (n = 10 individual plants).

| | WT | OEX1 | |
|---|---|---|---|
| | (5 mM NaCl) | (5 mM NaCl) | (200 mM NaCl) |
| Height (cm) | 124.0 ± 8.2 | 128.8 ± 9.5 | 107.6 ± 5.2 |
| Fresh Weight (g) | 1,270 ± 103 | 1,329 ± 110 | 1,123 ± 134 |
| (without fruit) | | | |
| Fruit per plant | 17.2 ± 1.3 | 17.8 ± .6 | 18.4 ± 1.5 |
| Fruit weight (g) | 119.5 ± 13.4 | 116.7 ± 9.0 | 105.7 ± 6.7 |
| Fruit water | 90.8 ± 3.2 | 90.2 ± 2.2 | 90.7 ± 2.3 |
| content (%) | | | |
| Solid solute | 4.2 ± 0.6 | 4.4 ± 0.7 | 4.2 ± 0.5 |
| content (⁰Brix) | | | |

TABLE II

| | | | | |
|---|---|---|---|---|
| SEQ | PROTEIN | | PROTEIN | |
| ID | NUMBER | PROTEIN | DESCRIPTION | |
| NO | (GI) | ACCESSION | (SPECIES) | SEQUENCE |

| | | | | | |
|---|---|---|---|---|---|
| 2 | NHX1 | AAD16946 | NHX1 Na+/H+ | 1 | MLDSLVSKLP SLSTSDHASV VALNLFVALL CACIVLGHLL EENRWMNESI TALLIGLGTG |
| | 4324597 | | exchanger | 61 | VTILLISKGK SSHLLVFSED LFFIYLLPPI IFNAGFQVKK KQFFRNFVTI MLFGAVGTII |
| | | | Arabidopsis | 121 | SCTIISLGVT QFFKKLDIGT FDLGDYLAIG AIFAATDSVC TLQVLNQDET PLLYSLVFGE |
| | | | thaliana | 181 | GVVNDATSVV VFNAIQSFDL THLNHEAAFH LLGNFLYLFL LSTLLGAATG LISAYVIKKL |
| | | | | 241 | YFGRHSTDRE VALMMLMAYL SYMLAELFDL SGILTVFFCG IVMSHYTWHN VTESSRITTK |
| | | | | 301 | HTFATLSFLA ETFIFLYVGM DALDIDKWRS VSDTPGTSIA VSSILMGLVM VGRAAFVFPL |
| | | | | 361 | SFLSNLAKKN QSEKINFNMQ VVIWWSGLMR GAVSMALAYN KFTRAGHTDV RGNAIMITST |
| | | | | 421 | ITVCLFSTVV FGMLTKPLIS YLLPHQNATT SMLSDDNTPK SIHIPLLDQD SFIEPSGNHN |
| | | | | 481 | VPRPDSIRGF LTRPTRTVHY YWRQFDDSFM RPVFGGRGFV PFVPGSPTER NPPDLSKA |
| 3 | 10716129 | BAB16380 | Na+/H+ | 1 | MAFGLSSLLQ NSDLFTSDHA SVVSMNLFVA LLCACIVLGH LLEENRWVNE SITALIIGLC |
| | | | exchanger | 61 | TGVVILLLSG GKSSHLLVFS EDLFFIYLLP PIIFNAGFQV KKKQFFVNFM TIMLFGAIGT |
| | | | Ipomoea nil | 121 | LISCSIISFG AVKIFKHLDI DFLDFGDYLA IGAIFAATDS VCTLQVLSQD ETPLLYSLVF |
| | | | | 181 | GEGVVNDATS VVLFNAIQSF DMTSFDPKIG LHFIGNFLYL FLSSTFLGVG IGLLCAYIIK |
| | | | | 241 | KLYFGRHSTD REVALMMLMS YLSYIMAELF YLSGILTVFF CGIVMSHYTW HNVTESSRVT |
| | | | | 301 | TRHSFATLSF VAETFIFLYV GMDALDIEKW KFVKNSQGLS VAVSSILVGL ILVGRAAFVF |
| | | | | 361 | PLSFLSNLAK KNSSDKISFR QQIIIWWAGL MRGAVSIALA YNKFTTSGHT SLHENAIMIT |
| | | | | 421 | STVTVVLFST VVFGLMTKPL INLLLPPHKQ MPSGHSSMTT SEPSSPKHFT VPLLDNQPDS |
| | | | | 481 | ESDMITGPEV ARPTALRMLL RTPTHTVHRY WRKFDDSFMR PVFGGRGFVP FVAGSPVEQS |
| | | | | 541 | PR |
| 4 | 14039961 | AAK53432 | Na+/H+ | 1 | MLSQLSSFFA SKMDMVSTSD HASVVSMNLF VALLRGCIVI GHLLEENRWM NESITALLIG |
| | | | Antiporter | 61 | LSTGIIILLI SGGKSSHLLV FSEDLFFIYL LPPIIFNAGF QVKKKQFFRN FITIILFGAV |
| | | | Suaeda | 121 | GTLVSFIIIS LGSIAIFQKM DIGSLELGDL LAIGAIFAAT DSVCTLQVLN QDETPLLYSL |
| | | | maritima | 181 | VFGEGVVNDA TSVVLFNAIQ NFDLTHIDHR IAFQFGGNFL YLFFASTLLG AVTGLLSAYV |
| | | | subsp. | 241 | IKKLYFGRHS TDREVALMML MAYLSYMLAE LFYLSGILTV FFCGIVMSHY TWHNVTESSR |
| | | | salsa | 301 | VTTKHAFATL SFVAEIFIFL YVGMDALDIE KWRFVSDSPG TSVAVSSILL GLHMVGRAAF |
| | | | | 361 | VFPFAFLMNL SKKSNSEKVT FNQQIVIWWA GLMKSAVSVA LAYNQFSRSG HTQLRGNAIM |
| | | | | 421 | ITSTITVVLF STMVFGLLTK PLILFMLPQP KHFTSASTVS DLGSPKSFSL PLLEDRQDSE |
| | | | | 481 | ADLGNDDEEA YPRGTIARPT SLRMLLNAPT HTVHHYWRRF DDYFMRPVFG GRGFVPFVPG |
| | | | | 541 | SPTEQSITNF VTENIS |
| 5 | 14211574 | BAB56105 | Na+/H+ | 1 | MAFDFGTLLG NVDRLSTSDH QSVVSINLFV ALICACIVIG HLLEENRWMN ESITALVIGS |
| | | | Antiporter | 61 | CTGIVILLIS GGKNSHILVF SEDLFFIYLL PPIIFNAGFQ VKKKSFFRNF STIMLFGALG |
| | | | Petunia x | 121 | TLISFIIISL GAIGIFKKMN IGSLEIGDYL AIGAIFSATD SVCTLQVLNQ DETPLLYSLV |
| | | | hybrida | 181 | FGEGVVNDAT SVVLFNAIQN FDLSHIDTGK AMELVGNFLY LFASSTALGV AAGLLSAYII |
| | | | | 241 | KKLYFGRHST DREVAIMILM AYLSYMLAEL FYLSAILTVF FSGIVHSHYT WHNVTESSRV |
| | | | | 301 | TTKHTFATLS FIAEIFIFLY VGMDALDIEK WKFVSDSPGI SVQVSSILLG LVLVGRAAFV |
| | | | | 361 | FPLSFLSNLT KKTPEAKISF NQQVTIWWAG LMRGAVSMAL AYNQFTRGGH TQLRANAIMI |
| | | | | 421 | TSTITVVLFS TVVFGLMTKP LIRILLPSHK HLSRMISSEP TTPKSFIVPL LDSTQDSEAD |
| | | | | 481 | LERHVPRPHS LRMLLSTPSH TVHYYWRKFD NAFMRPVFGG RGFVPFAPGS PTDPVGGNLQ |
| 6 | 14211578 | BAB56107 | Na+/H+ | 1 | MGFESVIKLA ASETDNLWSS GHGSVVAITL FVTLLCTCIV IGHLLEENRW MNESIIALII |
| | | | Antiporter | 61 | GLATGVIILL ISGGKSSHLL VFSEDLFFIY ALPPIIFNAG FQVKKKSFFR NFATIMMFGA |
| | | | Torenia | 121 | VGTLISFTII SLGTIAFFPK MNMRLGVGDY LAIGAIFAAT DSVCTLQVLS QDETPLLYSL |
| | | | hybrida | 181 | VFGEGVVNDA TSVVLFNAVQ NFDLPHMSTA KAFELVGNFF YLFATSTVLG VLTGLLSAYI |
| | | | | 241 | IKKLYFGRHS TDREVAIMIL MAYLSYMLAE LFDLSGILTV FFCGIVMSHY TWHNVTENSR |
| | | | | 301 | VTTKHTFATL SFVAEIFIFL YVGMDALDIE KWRFVSGSMT TSAAVSATLL GLVLLSRAAF |
| | | | | 361 | VFPLSFLSNL AKKSPLEKIS LRQQIIIWWA GLMRGAVSMA LAYKQFTREG LTVERENAIF |
| | | | | 421 | ITSTITIVLF STVVFGLMTK PLINLLIPSP KLNRSVSSEP LTPNSITIPL LGESQDSVAE |
| | | | | 481 | LFSIRGQTSQ GGEPVARPSS LRMLLTKPTH TVHYYWRKFD NAFMRPVFGG RGFVPYVPGS |
| | | | | 541 | PTERSVRNWE EETKQ |
| 7 | 14488270 | BAB60901 | Na+/H+ | 1 | MAFGLSSLLQ NSELFTSDHA SVVSMNLFVA LLCACIVLGH LLEENRWVNE SITALIIGLC |
| | | | exchanger | 61 | TGVVILLLSR GKSSHLLVFS EDLFFIYLLP PIIFNAGFQV KKKQFFVNFM TIMLFGAIGT |
| | | | Ipomoea | 121 | LISCSIISFG AVKIFKHLDI DFLDFGDYLA IGAIFAATDS VCTLQVLSQD ETPLLYSLVF |
| | | | tricolor | 181 | GEGVVNDATS VVLFNAIQSF DMTSFDPKIG LHFIGNFLYL FLSSTFLGVG IGLLCAYIIK |
| | | | | 241 | KLYFGRHSTD REVALMMLMS YLSYIMAELF YLSGILTVFF CGIVMSHYTW HNVTESSRVT |
| | | | | 301 | TRHSFATLSF VAETFIFLYV GMDALDIEKW KFVKNSQGLS VAVSSILVGL ILVGRAAFVF |
| | | | | 361 | PLSFLSNLAK KNSSDKISFR QQIIIWWAGL MRGAVSIALA YNKFTTSGHT SLHENAIMIT |
| | | | | 421 | STVTVVLFST VVFGLMTKPL INLLLPPHKQ IASGHSSMTT SEPSSPKHFA VPLLDNQHDS |
| | | | | 481 | ESDMITGPEV ARPTALRMLL RTPTHTVHRY WRKFDDSFMR PVFGGRGFVP FVAGSPAEQS |
| | | | | 541 | PR |
| 8 | 4585981 | AAD25617 | similar to | 1 | MISPVEHDPQ GQVKQQQAAG VGILLQIMML VLSFVLGHVL RRHRFHYLPE ASGSLLIGLI |
| | | | Na+/H+- | 61 | VGILANISDT ETSIRFCPPP SIPEFSLLSF PRSLVCSFYS VSGRGLISTK SSSSCFCCLP |
| | | | exchanging | 121 | SYYILCFNIC ISSFKFAAAM LCIMDVIFLD IIHLFEPSQV SVFNLNHSFL TLEPLLPLLS |
| | | | proteins | 181 | SELLSLQLLL VVCYLGGSMY LMYKLPFVEC LMFGALISAT DPVTVLSIFQ VLLLFLLLSV |
| | | | Arabidopsis | 241 | STGYKYSHDV GTDVNLYALV FGESVLNDAV SFYYLLRYWA LPFKTMSLVN RQSSSGEHFF |
| | | | thaliana | 301 | MVVIRFFETF AGSMSAGLAI SFLNSFYTVV FTLLILSEHI VNVMSLFSLF STSIHACRRC |
| | | | | 361 | WSLRHCFYTL HRNCNRRVMK RYTFSNLSEA SQSFVSSFFH LISSLAETFT FIYMGFDIAM |
| | | | | 421 | EQHSWSEVGA VNVFGCAYLV NLFRQENQKI PMKHQKALWY SGLRGAMAFA LALQSLHDLP |
| | | | | 481 | EGHGQIIFTA TTTIVVVTVT FVLLIGGSTG KMLEALEVVG DDLDDSMSEV NSRRSTLISL |
| | | | | 541 | NIGASSDEDT SSSGSRFKMK LKEFHKTGDG DGDGE |
| 9 | 8515714 | AAF76139 | putative | 1 | MTTVIDATMA YRFLEEATDS SSSSSSSKLE SSPVDAVLFV GMSLVLGIAS RHLLRGTRVP |
| | | | Na+/H+ | 61 | YTVALLVIGI ALGSLEYGAK HNLGKIGHGI RIWNEIDPEL LLAVFLPALL FESSFSMEVH |
| | | | antiporter | 121 | QIKRCLGQMV LLAVPGVLIS TACLGSLVKV TFPYEWDWKT SLLLGGLLSA TDPVAVVALL |
| | | | SOS1 | 181 | KELGASKKLS TIIEGESLMN DGTAIVVFQL FLKMAMGQNS DWSSIIKFLL KVALGAVGIG |
| | | | Arabidopsis | 241 | LAFGIASVIW LKFIFNDTVI EITLTIAVSY FAYYTAQEWA GASGVLTVMT LGHFYAAFAR |
| | | | thaliana | 301 | TAFKGDSQKS LHHFWEMVAY IANTLIFILS GVVIAEGILD SDKIAYQGNS WRFLFLLYVY |
| | | | | 361 | IQLSRVVVVG VLYPLLCRFG YGLDWKESII LVWSGLRGAV ALALSLSVKQ SSGNSHISKE |
| | | | | 421 | TGTLFLFFTG GIVFLTLIVN GSTTQFVLRL LRMDILPAPK KRILEYTKYE MLNKALRAFQ |
| | | | | 481 | DLGDDEELGP ADWPTVESYI SSLKGSEGEL VHHPHNGSKI GSLDPKSLKD IRMRFLNGVQ |
| | | | | 541 | ATYWEMLDEG RISEVTANIL MQSVDEALDQ VSTTLCDWRG LKPHVNFPNY YNFLHSKVVP |
| | | | | 601 | RKLVTYFAVE RLESACYISA AFLRAHTIAR QQLYDFLGES NIGSIVINES EKEGEEAKKF |
| | | | | 661 | LEKVRSSFPQ VLRVVKTKQV TYSVLNHLLG YIENLEKVGL LEEKEIAHLH DAVQTGLKKL |
| | | | | 721 | LRNPPIVKLP KLSDMITSHP LSVALPPAFC EPLKHSKKEP MKLRGVTLYK EGSKPTGVWL |
| | | | | 781 | IFDGIVKWKS KILSNNHSLH PTFSHGSTLG LYEVLTGKPY LCDLITDSMV LCFFIDSEKI |
| | | | | 841 | LSLQSDSTID DFLWQESALV LLKLLRPQIF ESVAMQELRA LVSTESSKLT TYVTGESIEI |
| | | | | 901 | DCNSIGLLLE GFVKPVGIKE ELISSPAALS PSNGNQSFHN SSEASGIMRV SFSQQATQYI |
| | | | | 961 | VETRARAIIF NIGAFGADRT LHRRPSSLTP PRSSSSDQLQ RSFRKEHRGL MSWPENIYAK |
| | | | | 1021 | QQQEINKTTL SLSERAMQLS IFGSMVNVYR RSVSFGGIYN NKLQDNLLYK KLPLNPAQGL |
| | | | | 1081 | VSAKSESSIV TKKQLETRKH ACQLPLKGES STRQNTMVES SDEEDEDEGI VVRIDSPSKI |
| | | | | 1141 | VFRNDL |
| 10 | 9857314 | BAB11940 | Na/H | 1 | MWSQLSSLLS GKMDALTTSD HASVVSMNLF VALLCGCIVI GHLLEENRWM NESITALLIG |
| | | | antiporter | 61 | LATGVVILLI SGGKSSHLLV FSEDLFFIYL LPPIIFNAGF QVKKKQFFRN FITIVLFGAV |
| | | | Nhx1 | 121 | GTLVSFTIIS LGALSIFKKL DIGTLELADY LAIGAIFAAT DSVCTLQVLN QDETPLLYSL |
| | | | Atriplex | 181 | VFGEGVVNDA TSVVLFNAIQ SFDLTRIDHR IALQFMGNFL YLFIASTILG AFTGLLSAYI |
| | | | gmelini | 241 | IKKLYFGRHS TDREVALMML MAYLSYMLAE LFYLSGILTV FFCGIVMSHY TWHNVTESSR |
| | | | | 301 | VTTKHAFATL SFVAEVFLFL YVGMDALDIE KWRFVSDSPG ISVAVSSILL GLVMVGRAAF |
| | | | | 361 | VFPLSWLMNF AKKSQSEKVT FNQQIVIWWA GLMRGAVSMA LAYNQFTRSG HTQLRGNAIM |
| | | | | 421 | ITSTISVVLF STMVFGLLTK PLIMFLLPQP KHFTSCSTVS DVGSPKSYSL PLLEGNQDYE |
| | | | | 481 | VDVGNGNHED TTEPRTIVRP SSLRMLLNAP THTVHHYWRK FDDSFMRPVF GGRGFVPFVP |
| | | | | 541 | GSPTEQSTNN LVDRT |
| 11 | NHA1 | NP_013239 | Putative | 1 | MAIWEQLEVS KAHVAYACVG VFSSIFSLVS LYVKEKLYIG ESTVAGIFGL IVGPVCLNWF |
| | 6323167 | | Na+/H+ | 61 | NPLKWGNSDS ITLEITRIVL CLQIFAVAVE LPRKYMLKHW VSVTMLLLPV MTAGWLIIGL |
| | | | antiporter, | 121 | FVWILIPGLN FSASLLISAC ITATDPILAQ SVVSGKFAQR VPGHLRNLLS AESGCNDGMA |
| | | | Nhalp | 181 | FPFLFLSMNL ILHPGNGREI VKDWICVTIL YECLFGCLLG CFIGYVGRIT IRFAEKKNII |
| | | | Saccharo- | 241 | DRESFLAFYV VLAFMCAGFG SILGVDDLLV SFAAGATFAW DGWFSQKTQE SNVSTVIDLL |
| | | | myces | 301 | LNYAYFIYFG AIIPWSQFNN GEIGTNVWRL IILSIVVIFL RRIPAVMILR PLIPDIKSWR |
| | | | cerevisiae | 361 | EALFVGHFGP IGVGAIFAAI LARGELESTF SDEPTPLNVV PSKEESKHWQ LIACIWPITC |
| | | | | 421 | FFIVTSIIVH GSSVAIITLG RHLNTITLTK TFTTHTTNGD NGKSSWMQRL PSLDKAGRSF |
| | | | | 481 | SLHRMDTQMT LSGDEGEAEE GGGRKGLAGG EDEEGLNNDQ IGSVATSGIP ARPAGGMPRR |
| | | | | 541 | RKLSRKEKRL NRRQKLRNKG REIFSSRSKN EMYDDDELND LGRERLQKEK EARAATFALS |
| | | | | 601 | TAVNTQRNEE IGMGGDEEED EYTPEKEYSD NYNNTPSFES SERSSSLRGR TYVPRNRYDG |
| | | | | 661 | EETESEIESE DEMENESERS MASSEERRIR KMKEEEMKPG TAYLDGNRMI IENKQGEILN |
| | | | | 721 | QVDIEDRNEA RDDEVSVDST AHSSLTTTMT NLSSSSGGRL KRILTPTSLG KIHSLVDKGK |
| | | | | 781 | DKNKNSKYHA FKIDNLLIIE NEDGDVIKRY KINPHKSDDD KSKNRPRNDS VVSRALTAVG |
| | | | | 841 | LKSKANSGVP PPVDEEKAIE GPSRKGPGML KKRTLTPAPP RGVQDSLDLE DEPSSEEDLG |
| | | | | 901 | DSYNMDDSED YDDNAYESET EFERQRRLNA LGEMTAPADQ DDEELPPLPV EAQTGNDGPG |
| | | | | 961 | TAEGKKKQKS AAVKSALSKT LGLNK |
| 12 | NHX1 | NP_010744 | Required | 1 | MLSKVLLNIA FKVLLTTAKR AVDPDDDDEL LPSPDLPGSD DPIAGDPDVD LNPVTEEMFS |
| | 6320663 | | for intra- | 61 | SWALFIMLLL LISALWSSYY LTQKRIRAVH ETVLSIFYGM VIGLIIRMSP GHYIQDTVTF |
| | | | cellular | 121 | NSSYFFNVLL PPIILNSGYE LNQVNFFNNM LSILIFAIPG TFISAVVIGI ILYIWTFLGL |
| | | | sequestra- | 181 | ESIDISFADA MSVGATLSAT DPVTILSIFN AYKVDPKLYT IIFGESLLND AISIVMFETC |
| | | | tion of | 241 | QKFHGQPATF SSVFEGAGLF LMTFSVSLLI GVLIGILVAL LLKHTHIRRY PQIESCLILL |
| | | | Na+; Nhxlp | 301 | IAYESYFFSN GCHMSGIVSL LFCGITLKHY AYYNMSRRSQ ITIKYIFQLL ARLSENFIFI |
| | | | Saccharo- | 361 | YLGLELFTEV ELVYKPLLII VAAISICVAR WCAVFPLSQF VNWIYRVKTI RSMSGITGEN |
| | | | myces | 421 | ISVPDEIPYN YQMMTFWAGL RGAVGVALAL GIQGEYKFTL LATVLVVVVL TVIIFGGTTA |
| | | | cerevisiae | 481 | GMLEVLNIKT GCISEEDTSD DEFDIEAPRA INLLNGSSIQ TDLGPYSDNN SPDISIDQFA |
| | | | | 541 | VSSNKNLPNN ISTTGGNTFG GLNETENTSP NPARSSMDKR NLRDKLGTIF NSDSQWFQNF |
| | | | | 601 | DEQVLKPVFL DNVSPSLQDS ATQSPADFSS QNH |
| 13 | NHX2 | NP_187154 | NHX2 Na+/H+ | 1 | MTMFASLTSK MLSVSTSDHA SVVSLNLFVA LLCACIVIGH LLEENRWMNE SITALLIGLG |
| | 15229877 | | exchanger | 61 | TGVVILLISR GKNSHLLVFS EDLFFIYLLP PIIFNAGFQV KKKQFFRNFV TIMAFGAIGT |
| | | | Arabidopsis | 121 | VVSCTIISLG AIQFFKKLDI GTFDLGDFLA IGAIFAATDS VCTLQVLNQD ETPLLYSLVF |
| | | | thaliana | 181 | GEGVVNDATS VVLFNAIQSF DLTHLNHEAA FQFLGNFFYL FLLSTGLGVA TGLISAYVIK |
| | | | | 241 | KLYFGRHSTD REVALMMLMA YLSYMLAELF ALSGILTVFF CGIVMSHYTW HNVTESSRIT |
| | | | | 301 | TKHAFATLSF LAETFIFLYV GMDALDIEKW RFVSDSPGTS VAVSSILMGL VMLGRAAFVF |
| | | | | 361 | PLSFLSNLAK KHQSEKISIK QQVVIWWAGL MRGAVSMALA YNKFTRSGHT ELRGNAIMIT |
| | | | | 421 | STITVCLFST MVFGMLTKPL IRYLMPHQKA TTSTTSMLSD DSTPKSIHIP LLDGEQLDSF |
| | | | | 481 | ELPGSHQDVP RPNSLRGFLM RPTRTVHYYW RQFDDAFMRP VFGGRGFVPF VPGSPTERSS |
| | | | | 541 | HDLSKP |
| 14 | NHX3 | NP_200358 | NHX3 Na+/H+ | 1 | MSIGLTEFVT NKLAAEHPQV IPISVFIAIL CLCLVIGHLL EENRWVNESI TAILVGAASG |
| | 15240159 | | exchanger | 61 | TVILLISKGK SSHILVFDEE LFFIYLLPPI IFNAGFQVKK KKFFHNFLTI MSFGVIGVFI |
| | | | Arabidopsis | 121 | STVIISFGTW WLFPKLGFKG LSARDYLAIG TIFSSTDTVC TLQILHQDET PLLYSLVFGE |
| | | | thaliana | 181 | GVVNDATSVV LFNAVQKIQF ESLTGWTALQ VFGNFLYLFS TSTLLGIGVG LITSFVLKTL |
| | | | | 241 | YFGRHSTTRE LAIMVLMAYL SYMLAELFSL SGILTVFFCG VLMSHYASYN VTESSRITSR |
| | | | | 301 | HVFAMLSFIA ETFIFLYVGT DALDFTKWKT SSLSFGGTLG VSGVITALVL LGRAAFVFPL |
| | | | | 361 | SVLTNFMNRH TERNESITFK HQVIIWWAGL MRGAVSIALA FKQFTYSGVT LDPVNAAMVT |
| | | | | 421 | NTTIVVLFTT LVFGFLTKPL VNYLLPQDAS HNTGNRGKRT EPGSPKEDAT LPLLSFDESA |
| | | | | 481 | STNFNRAKDS ISLLMEQPVY TIHRYWRKFD DTYMRPIFGG PRRENQPEC |
| 15 | NHX4 | NP_187288 | NHX4 Na+/H+ | 1 | MVIGLSTMLE KTEALFASDH ASVVSMNLFV ALLCACIVLG HLLEETRWMN ESITALIIGS |
| | 15230706 | | exchanger | 61 | CTGIVILLIS GGKSSRILVF SEDLFFIYLL PPIIFNAGFQ VKKKQFFRNF MTIMLFGAIG |
| | | | Arabidopsis | 121 | TLISFVIISF GAKHLFEKMN IGDLTIADYL AIGAIFSATD SVCTLQVLNQ DETPLLYSLV |
| | | | thaliana | 181 | FGEGVVNDAT SVVLFNAIQR FDLTNINSAI ALEFAGNFFY LFILSTALGV AAGLLSAFVI |
| | | | | 241 | KKLYIGRHST DREVALMMLL AYLSYMLAEL FHLSSILTVF FCGIVMSHYT WHNVTDKSKV |
| | | | | 301 | TTKHTFAAMS FLAEIFIFLY VGMDALDIEK WDVVRNSPGQ SIGVSSILLG LILLGRAAFV |
| | | | | 361 | FPLSFLSNLT KSSPDEKIDL KKQVTIWWAG LMRGAVSMAL AYNQFTTSGH TKVLGNAIMI |
| | | | | 421 | TSTITVVLFS TVVFGLLTKP LVKHLQPSSK QSSTTALQIT LRSSFHDPIL HEPLLSTQGQ |
| | | | | 481 | SEYDPEQHVS FRMFWKSPSR AIHHYWRKFD NAVMRRIFGG RGVSPVVPGS PIENSVPQWS |
| | | | | 541 | EEVENKEQNG EP |
| 16 | NHX5 | NP_175839 | NHX5 Na+/H+ | 1 | MEEVMISPVE HDPQGQVKQQ QAAGVGILLQ IMMLVLSFVL GHVLRRHRFH YLPEASGLIV |
| | 30695721 | | exchanger | 61 | GILANISDTE TSIRFCPPPS IPEFSLLSFP RSLKPFFSNF GAIVTFAIIG TFVASVVTGG |
| | | | Arabidopsis | 121 | LVYLGGSMYL MYKLPFVECL MFGALISATD PVTVLSIFQD VGTDVNLYAL VFGESVLNDA |
| | | | thaliana | 181 | VSFYYLLRYW ALPFKFFETF AGSMSAEHLF KYAGLDTENL QNLECCLFVL FPYFSYMLAE |
| | | | | 241 | GVGLSGIVSI LFTGIVMKRY TFSNLSEASQ SFVSSFFHLI SSLAETFTFI YMGFDIAMEQ |
| | | | | 301 | HSWSHVGFIL FSIVSSFTDR QAVNVFGCAY LVNLFRQENQ KIPMKHQKAL WYSGLRGAMA |
| | | | | 361 | FALALQSLHD LPEGHGQIIF TATTTIVVVT VLLIGGSTGK MLEALEVVGD DLDDSMSEGF |
| | | | | 421 | EESDHQYVPP PFSIGASSDE DTSSSGSRFK MKLKEFHKTT TSFTALDKNF LTPFFTTNSG |
| | | | | 481 | DGDGDGE |
| 17 | NHX6 | NP_178079 | NHX6 Na+/H+ | 1 | MSSELQISPA IHDPQGQEKQ QQAAGVGILL QIMMLVLSFV LGHVLRRHKF YYLPEASASL |
| | 22330742 | | exchanger | 61 | LIGLIVGGLA NISNTETSIR FVELFLISFF RHGSISTMSS SFCFCCLPSY YILKIEYLGG |
| | | | Arabidopsis | 121 | VMFLMYRLPF VECLMFGSLI SATDPVTVLS IFQELGSDVN LYALVFGESV LNDADEIVTL |
| | | | thaliana | 181 | LIRSFSFLCC FWQMAISLYR TMSLVRSHSS GQNFFMVIVR FLETFVGSMS AAMKYFILMY |
| | | | | 241 | SLLLSVYRTW SAVSSYFFHI SRNKTLLFYT SYVSIYFTLI EIVQFVMKHY TYSNLSANSQ |
| | | | | 301 | RFVSAFFHLI SSLAETFVFI YMGFDIAMEK HSWAANVFGC GYLVNLARPA HRKIPMTHQK |
| | | | | 361 | ALWYSGKILL CVPLSSYCFY SSVINTKICG FCIGLRGAMA FALALQSVHD LPEGHGQTIF |
| | | | | 421 | TATTAIVVLT VLLIGGSTGT MLEALEVVGD SHDTSLGDGF EVVNSRYMTS YDDEDTPPGS |
| | | | | 481 | GFRTKLREFH KSAASFTELD RNYLTPFFTS NNGDYDDEGN MEQHHGNNII L |
| 18 | NHX7 | NP_178307 | NHX7 Na+/H+ | 1 | MTSIIGAALP YKSPEKAIAS SSYSAENDSS PVDAVIFAGT SLVLGTACRY LFNGTRVPYT |
| | 22325422 | | exchanger | 61 | VVLLVIGIFL GSLEYGTKHN LGKLGHGIRI WNGINPDLLL AVFLPVLLFE SSFSMDVHQI |
| | | | Arabidopsis | 121 | KRCMGQMVLL AGPGVLISTF CLGALIKLTF PYNWDWKTSL LLGGLLGATD PVAVVALLKE |
| | | | thaliana | 181 | LGASKKMTTL IDGESLMNDG VSVVVFQLFF KMVMGHNSDW GSIIKFLVQN SFGAVGIGLA |
| | | | | 241 | FGIASVFWLK FIFNDTVAQI TVTLSASYFA YYTAQEWAGV SGILTVMILG MFFAAFARTA |
| | | | | 301 | FKGDSHQSLH HFWYFTTQEM AAYIANTLVF MLSGVIIAES VLSGQTISYK AIKWKFISQF |
| | | | | 361 | RYGNKAVLQF LFLTGGIVFL TLVVNGSTTQ LLLHLLRMDT LTATKKRILE YTKFEMMKTA |
| | | | | 421 | LKAFENLGDD EELGSADWPT VIRHISSLKD LEGRQVNPHD GYEAGSLDPT NIMDIRVQAA |
| | | | | 481 | YWEMLDDGRI TQCTANVLMQ SVDEALDLVS TSSLSDWRGL EPRVHFPNYY KFLQSKIIPH |
| | | | | 541 | KLVTHLIVER LESACYISSA FLRAHRIARQ QLHIFLGNSN IASTVINESE VEGEEAKQFL |
| | | | | 601 | EDVRDSFPQV LSVLKTRQVT HYVLNHLNGY IKNLEKVGLL EGKEVSHLHD VVQSDLKKLL |
| | | | | 661 | RHPPSLKLPN VDDLITSNPL LKDRSSFRSL AIGETDA |
| 19 | NHX8 | NP_172918 | NHX8 Na+/H+ | 1 | MTSIIGAALP YKSPEKAIAS SSYSAENDSS PVDAVIFAGT SLVLGTACRY LFNGTRVPYT |
| | 15223849 | | exchanger | 61 | VVLLVIGIFL GSLEYGTKHN LGKLGHGIRI WNGINPDLLL AVFLPVLLFE SSFSMDVHQI |
| | | | Arabidopsis | 121 | KRCMGQMVLL AGPGVLISTF CLGALIKLTF PYNWDWKTSL LLGGLLGATD PVAVVALLKE |
| | | | thaliana | 181 | LGASKKMTTL IDGESLMNDG VSVVVFQLFF KMVMGHNSDW GSIIKFLVQN SFGAVGIGLA |
| | | | | 241 | FGIASVFWLK FIFNDTVAQI TVTLSASYFA YYTAQEWAGV SGILTVMILG MFFAAFARTA |
| | | | | 301 | FKGDSHQSLH HFWYFTTQEM AAYIANTLVF MLSGVIIAES VLSGQTISYK AIKWKFISQF |
| | | | | 361 | RYGNKAVLQF LFLTGGIVFL TLVVNGSTTQ LLLHLLRMDT LTATKKRILE YTKFEMMKTA |
| | | | | 421 | LKAFENLGDD EELGSADWPT VIRHISSLKD LEGRQVNPHD GYEAGSLDPT NIMDIRVQAA |
| | | | | 481 | YWEMLDDGRI TQCTANVLMQ SVDEALDLVS TSSLSDWRGL EPRVHFPNYY KFLQSKIIPH |
| | | | | 541 | KLVTHLIVER LESACYISSA FLRAHRIARQ QLHIFLGNSN IASTVINESE VEGEEAKQFL |
| | | | | 601 | EDVRDSFPQV LSVLKTRQVT HYVLNHLNGY IKNLEKVGLL EGKEVSHLHD VVQSDLKKLL |
| | | | | 661 | RHPPSLKLPN VDDLITSNPL LKDRSSFRSL AIGETDA |
| 20 | 15982204 | CAC84522 | Na+/H+ | 1 | MGLDAVARLG VSILSDGDQV SVDSITLFVA LLCGCIVIGH LLEESRWIND SITTLVIGLS |
| | | | antiporter, | 61 | TGGIILLTTK GKSSHLLEFD EQLFFIYVLP PIIFNAGFQV KKKQFFRNFV TIMLFGAVGT |
| | | | isoform 1 | 121 | LISFSIISFG AKELLGKLDI GFLELRDYLA IGAIFSATDS VCTLQALNQD ETPRLYSLVF |
| | | | Lycopersi- | 181 | GEGVVNDATS VVLFNAIQKL DLSHINSRAA LVFTGNFLYL FLASTFLGVL IGLLSAYLIK |
| | | | con | 241 | KIYLGRHSTD REVALMILMA YLSYVMAELF DLSGILTVFI CGIVMSHYTW HNVTFNSKVT |
| | | | esculentum | 301 | TRHAFATLSF IAEIFIFLYV GMDALDIEKW RFVKDSPGKS VGVSAALLGL VLVGRACFVF |
| | | | | 361 | PLSLFSNCLK RSEHDKFGLK LQVTIWWAGL MRGSVSMALA YNQFTRFGHT QQPGNAVMIT |
| | | | | 421 | STITIVLFST VVFGLITKPL VRFLLPSSQG FNNLISSEQS FARPLLTNEQ ELELEMGNVD |
| | | | | 481 | PVRPSGLSIL LKEPSYTIHN HWRRFDDAFM RPLFGGRGFV PDAPELSKGG CDQY |
| 21 | 15982206 | CAC83608 | Na+/H+ | 1 | MEDHLQISPA GAKAIPGKEQ QAAGYGILLQ IMMLVLSFVI GHVLRRRHFY YIPEASASLL |
| | | | antiporter, | 61 | IGLIVGGLAN VSDTETSIRA WFNFHEEFFF LFLLPPIIFQ SGFSLSPKPF FSNFGAIITF |
| | | | isoform 2 | 121 | AILGTFIASF VTGILVYLGG VTYLMYRLPF VECLMFGALI SATDPVTVLS IFQELGTDVN |
| | | | Lycopersi- | 181 | LYALVFGESV LNDAMAISLY RTMSLVRSHM STDQNYFMIT IRFVETFMGS LSAGVGVGFV |
| | | | con | 241 | SALLFKYAGL DIDNLQNLES CLFVLFPYFS YMLAEGLGLS GIVSILFTGV VMKRYTYPNL |
| | | | esculentum | 301 | SESSQRFVSA FFHLISSLAE TFVFIYMGFD IAMEKHSWSH VGFIFFSILF IVIARAANVF |
| | | | | 361 | GCAYLVNLVR PPHQKIPAKH QKALWYSGLR GAMAFALALQ PVHDLPEGHG QAIFTATTAI |
| | | | | 421 | VVLTVLIIGG SAGTMLEALE VVGDGQSGSM DETFEGNNGY IAPSYRDESY DGEPSSGNRF |
| | | | | 481 | RMKLKEFHKS TTSFSALDKN YLTPFFTTQG GDEDEDEPIM HSSRRAGYDG H |
| 22 | 5731737 | BAA83337 | OsNHX1 | 1 | MGMEVAAARL GALYTTSDYA SVVSINLFVA LLCACIVLGH LLEENRWVNE SITALIIGLC |
| | | | Oryza | 61 | TGVVILLMTK GKSSHLFVFS EDLFFIYLLP PIIFNAGFQV KKKQFFRNFM TITLFGAVGT |
| | | | sativa | 121 | MISFFTISIA AIAIFSRMNI GTLDVGDFLA IGAIFSATDS VCTLQVLNQD ETPFLYSLVF |
| | | | (japonica | 181 | GEGVVNDATS IVLFNALQNF DLVHIDAAVV LKFLGNFFYL FLSSTFLGVF AGLLSAYIIK |
| | | | cultivar- | 241 | KLYIGRHSTD REVALMMLMA YLSYMLAELL DLSGILTVFF CGIVMSHYTW HNVTESSRVT |
| | | | group) | 301 | TKHAFATLSF IAETFLFLYV GMDALDIEKW EFASDRPGKS IGISSILLGL VLIGRAAFVF |
| | | | | 361 | PLSFLSNLTK KAPNEKITWR QQVVIWWAGL MRGAVSIALA YNKFTRSGHT QLHGNAIMIT |
| | | | | 421 | STITVVLFST MVFGMMTKPL IRLLLPASGH PVTSEPSSPK SLHSPLLTSM QGSDLESTTN |
| | | | | 481 | IVRPSSLRML LTKPTHTVHY YWRKFDDALM RPMFGGRGFV PFSPGSPTEQ SHGGR |
| 23 | 14211576 | BAB56106 | Na+/H+ | 1 | MAFDFGTLLG KMNNLTTSDH QSVVSVNLFV ALICACIVIG HLLEENRWMN ESITALVIGS |
| | | | antiporter, | 61 | CTGVIILLIS GGKNSHILVF SEDLFFIYLL PPIIFNAGFQ VKKKSFFRNF STIMLFGAVG |
| | | | Nierem- | 121 | TLISFIIISA GAIGIFKKMD IGHLEIGDYL AIGAIFAATD SVCTLQVLNQ EETPLLYSLV |
| | | | bergia | 181 | FGEGVVNDAT SVVLFNAVQN FDLSHISTGK ALQLIGNFLY LFASSTFLGV AVGLLSAFII |
| | | | caerulea | 241 | KKLYFGRHST DREVAIMILM AYLSYMLAEL FYLSGILTVF FCGIVMSHYT WHNVTESSRV |
| | | | | 301 | TTKHTFATLS FIAEIFIFLY VGMDALDIEK WKFVSDSPGT SIKVSSILLG LVLVGRGAFV |
| | | | | 361 | FPLSFLSNLT KKNPEDKISF NQQVTIWWAG LMRGAVSMAL AYNQFTRGGH TQLRANAIMI |
| | | | | 421 | TSTITVVLFS TVVFGLMTKP LILLLLPSQK HLIRMISSEP MTPKSFIVPL LDSTQDSEAD |
| | | | | 481 | LGRHVPRPHS LRMLLSTPSH TVHYYWRKFD NAFMRPVFGG RGFVPFVPGS PTEPVEPTEP |
| | | | | 541 | RPAESRPTEP TDE |
| 24 | 15812035 | AAK27314 | Na+/H+ | 1 | MDQAISSVVR KLQMVNTSDH NSVVSINIFV ALPCASIVIG HLLEESRWMN ESITALLIGV |
| | | | exchanger | 61 | CAGVIILLTT GGKSSHLFVF SEDLFFIYVL PPIIFNAGFQ VKKKQFFRNF ITIMLFGAIG |
| | | | Citrus x | 121 | TLVSCTIISL GVIQFFKKLD IGTLDIGDYL AIGAIFAATD SVCTLQVLNQ DDTPLLYSLV |
| | | | paradisi | 181 | FGEGVVNDAT SVVLFNAIQS FDLTHINTRS AFQFIGNFLY LFFTSTLLGV IGGLLSAYVI |
| | | | | 241 | KKLYFGRHST DREVAIMVLM AYLSYMLAEL FYLSGILTVF FCGIVMSHYT WHNVTESSRV |
| | | | | 301 | TTKHTFATLS FVAEIFTFLY VGMDALDIEK WRFVKGSPGT SVAASAMLMG LIMAGRAAFV |
| | | | | 361 | FPLSFLTNLA KKSPTEKISI KQQVIIWWAG LMRGAVSMAL AYNQFTRSGH TQLRENAIMI |
| | | | | 421 | TSTITVVLFS TVVFGLMTEP LIRLLLPHPK HTTNHILSDP STPKSLSQPL LEEGQQDSYA |
| | | | | 481 | DLVGPTVPRP GSLRALLTTP THTVHYYWRK FDDAFMRPVF GGRGFAPFVP GSPTERSVRG |
| | | | | 541 | GQ |
| 25 | 15027833 | AAK76737 | Na+/H+ | 1 | MGLDLGALAL KYTGLAVSDH DSIVAINIFI ALLCGCIVFG HLLEGNRWVN ESTTALVLGL |
| | | | antiporter | 61 | ITGGVILICT KGVNSRILIF SEDIFFIYLL PPIIFNAGFQ VKKKQFFRNF ATIILFGAAG |
| | | | Triticum | 121 | TLISFVIITF GAMGLFSKLD VGPLELGDYL AIGAIFSATD SVCTLQVLNQ DEAPLLYSLV |
| | | | aestivum | 181 | FGEGVVNDAT SVVLFNAIQN IDINHFDVFV LLQFIGKFLY LFFTSTVLGV AAGLLSAYII |
| | | | | 241 | KKLCFARHST DREVAIMILM AYLSYMLSML LDLSGILTVF FCGIVMSHYT WHNVTESSRV |
| | | | | 301 | TTKHTFATLS FIAEIFLFLY VGMDALDIDK WKLASSSPKK PIALSAVILG LVMVGRAAFV |
| | | | | 361 | FPLSFLSNLS KKESHPKISF NQQVIIWWAG LMRGAVSIAL AYNKFTTSGH TAVRVNAVMI |
| | | | | 421 | TSTIIVVLFS TMVFGLLTKP LINLLIPPRP GTAADISSQS FLDPLTASLL GSDFDVGQLT |
| | | | | 481 | PQTNLQYLLT MPTRSVHRVW RKFDDKFMRP MFGGRGFVPF VPGSPIERSV HGPGLLGTVT |
| | | | | 541 | EAEDRS |
| 26 | 28575021 | AAK76738 | Na+/H+ | 1 | MGYQVVAAQL ARLSGALGTS DHASVVSITL FVALLCACIV LGHLLEENRW LNESITALII |
| | | | antiporter | 61 | GLCTGVVILM TTKGKSSHVL VFSEDLFFIY LLPPIIFNAG FQVKKKQFFR NFMAITLFGA |
| | | | Triticum | 121 | VGTMMSFFTI SLAAIAIFSR MNIGTLDVSD FLAIGAIFSA TDSVCTLQVL NQDETPFLYS |
| | | | aestivum | 181 | LVFGEGVVND ATSVVLFNAL QNFDPNQIDA IVILKFLGNF CYLFVSSTFL GVFTGLLSAY |
| | | | | 241 | VIKKLYIGRH STDREVALVM LMAYLSYMLA ELLDLSGILT VFFCGIVMSH YTWHNVTESS |
| | | | | 301 | RVTTKHAFAT LSFIAETFLF LYVGMDALDI EKWKFASDSP GKSIGISSIL LGLVLVGRAA |
| | | | | 361 | FVFPLSFLSN LTKKTELEKI SWRQQIVIWW AGLMRGAVSI ALAYNKFTRS GHTQLHGNAI |
| | | | | 421 | MITSTITVVL FSTMLFGILT KPLIRFLLPA SSNGAASDPA SPKSLHSPLL TSQLGSDLEA |
| | | | | 481 | PLPIVRPSSL RMLITKPTHT IHYYWRKFDD ALMRPMFGGR GFVPYSPGSP TDPNVLVE |
| 27 | 31580736 | AAP55209 | Na+/H+ | 1 | MGLDLGALAL KYTGLAVSDH DSIVAINIFI ALLCGCIVFG HLLGGNRWVN ESTAALVLGL |
| | | | antiporter | 61 | ITGGVILICT KGVNSRILIF SEDIFFIYLL PPIIFNAGFQ VKKKQFFRNF ATIILFGAAG |
| | | | Triticum | 121 | TLISFVIITF GAMGLFSKLD VGPLELGDYL AIGAIFSATD SVCTLQVLNQ DEAPLLYSLV |
| | | | aestivum | 181 | FGEGVVNDAT SVVLFNAIQN IDINHFDVFG LLQFIGKFLY LFFTSTVLGV AAGLLSAYII |
| | | | | 241 | KKLCFARHST DREVAIMILM AYLSCMLSML LDLSGILTVF FCGIVMSHYT WHNVTESSRV |
| | | | | 301 | TTKHTFATLS FIAEIFLFLY VGMDALDIDK WKLASSSPKK PIALSAVILG LVMVGPAAFV |
| | | | | 361 | FPLSFLSNLS KKESHPKISF NQQVIIWWAG LMRGAVSIAL AYNKFTTSGH TAVRVNAVMI |
| | | | | 421 | TSTIIVVLFS TMVFGLLTKP LINLLIPPRP GTAADISSQS FLDPLTASLL GSDFDVGQLT |
| | | | | 481 | PQTNLQYLLT MPTRSAHRVW RKFDDKFMRP MFGGRGFVPF VPGSPIERSV HGPGLLGTVT |
| | | | | 541 | EAEDRS |
| 28 | 30172039 | AAP20428 | Na+/H+ | 1 | MGLGVVAELV RLGVLSSTSD HASVVSINLF VALLCACIVL GHLLEENRWV NESTALIVGL |
| | | | antiporter | 61 | GTGTVILMIS RGVVIHVLVF SEDLFFFYLL PPIIFNAGFQ VKKKQFFRNF ITITLFGAVG |
| | | | NHX1 | 121 | TLISFTVISL GALGLISRLN IGALELGDYL ALGAIFSATD SVCTLQVLSQ DETPFLYSLV |
| | | | Zea mays | 181 | FGEGVVNDAT SVVVFNALQN FDITHIDAEV VFHLLGNFFY LFLLSTVLGV ATGLISALVI |
| | | | subsp. | 241 | KKLYFGRHST DREVALMMLM AYLSYMLAEL FALSGILTVF FGCIVMSHYT WHNVTESSRI |
| | | | mays | 301 | TTKHAFATLS FLAETFLFLY VGMDALDIDK WRSVSDTPGK SLAISSILMG LVMVGRAAFV |
| | | | | 361 | FPLSFLSNLA KKTEHEKISW KQQVVIWWAG LMRGAVSMAL AYKKFTRAGH TQVRGNAIMI |
| | | | | 421 | TSTIIVVLFS TMVFGLLTKP LINLLIPHRN ATSMLSDDSS PKSLHSPLLT SQLGSDLEEP |
| | | | | 481 | TNIPRPSSIR GEFLTMTRTV HRYWRKFDDA FMRPMFGGRG FVPFVPGSPT ERNPPDLSKA |
| 29 | 30172041 | AAP20429 | Na+/H+ | 1 | MGLGVDAETV RLGVLSSTSD HASVVSNNFF VALLCACIVL GHLLEENRMV NESITALLVG |
| | | | antiporter | 61 | LGTGTVILMI SRGVSIHVLV FSEDLFFIYL LPPIIFNAGF QVKKKQFFRN FITIILFGAI |
| | | | NHX2 | 121 | GTLISFVIIS LGAMGLFKKL DVGPLELGDY LAIGAIFSAT DSVCTLQVLN QDETPLLYSL |
| | | | Zea mays | 181 | VFGEGVVNDA TSIVVFNALQ NFDITHINAE VVFHLLGNFL YLFLLSTVLG VATGLISALV |
| | | | subsp. | 241 | IKKIYFGRHS TDREVALMML MAYLSYMLAE LFALSGILTV FFGCIVMSHY TWHNVTESSR |
| | | | mays | 301 | ITTKHAFATL SFLAETFIFL YVGMDALDIE KWRSVSDTPG KSIAISSILM GLVMLGRAAF |
| | | | | 361 | VFPLSFLSNL AKKNEHEKIS WKQQVVIWWS GLMRGAVSMA LAYNKFTRAG HTEVRGNEIM |
| | | | | 421 | ITSTITVVLF STVVFGLLTK PLIRLLMPHR HLTMLSDDST PKSLHSPLLT SQLGSSIEEP |
| | | | | 481 | TQIPRPTNIR GEFTTMTRTV HRYWRKFDDK FMRPMFGGRG FVPFVPGSPT ERNPHDLSKP |
| 30 | 32396168 | AAP20430 | Na+/H+ | 1 | MSIGLTAETV TNKLASAEHP QVVPNSVFIA LLCLCLVIGH LLEENRWVNE SITAILVGAA |
| | | | antiporter | 61 | TGTVILLISK GKSSHILVFD EELFFIYLLP PIIFNAGFQV KKKQFFRNFI TIILFGAIGT |
| | | | NHX3 | 121 | LISFVIISLG AMGLFKKLDV GPLELGDYLA IGAIFSATDS VCTLQVLNQD ETPLLYSLVF |
| | | | Zea mays | 181 | GEGVVNDATS VVLFNAVQKI DFEHLTGEVA LQVFGNFLYL FSTSTVLGIA TGLITAFVLK |
| | | | subsp. | 241 | TLYFGRHSTT RELAIMVLMA YLSFMLAELF SLSGIITVFF CGVLMSHVTW HNVTESSRIT |
| | | | mays | 301 | SRHVFAMLSF IAETFLFLYV GTDALDFTKW KTSSLSFGKS LGVSSVLLGL VLVGRAAFVF |
| | | | | 361 | PLSFLSNLSK KHPGEKITIR QQVVIWWAGL MRGAVSIALA FNKFTRAGHT QVRGNAIMIT |
| | | | | 421 | STIIVVLFST VVFGLLTKPL INLLIPHRNA TSMLSDDSSP KSLHSPLLTS QLISSIEEPT |
| | | | | 481 | QIPRPTNIRG EFMTMTRTVH RYWRKFDDKF MRPMFGGRGF VPFVPGSPTE RSSPDLSKA |
| 31 | 32396170 | AAP20431 | Na+/H+ | 1 | MGYQVVAAQL KLASSADHAS VVIITLFVAL LCACIVLGHL LEENRWLNES ITALIIGLGT |
| | | | antiporter | 61 | GVVILLISRG KNSRLLVFSE DLFFIYLLPP IIFNAGFQVK KKQFFRNFMT ITLFGAVGTM |
| | | | NHX4 | 121 | ISFFTISLGA IATFSRMSIG TLDVGDFLAI GAIFSATDSV CTLQVLHQDE TPFLYSLVFG |
| | | | Zea mays | 181 | EGVVNDATSV VLFNAVQKIQ FTRINAWTAL QLIGNFLYLF STSTLLGIGT GLITAFVLKK |
| | | | subsp. | 241 | LYFGRHSTTR ELAIMILMAY LSYMLAELFS LSGLLTVFFC GVLMSHVTWH NVTESSRTTS |
| | | | mays | 301 | RHVFATLSFI SETFIFLYVG MDALDFEKWK TSSLSFGGTL GVSGVLMGLV MLGRAAFVFP |
| | | | | 361 | LSFLSNLAKK HQSEKISFRM QVVIWWAGLM RGAVSMALAL NKFTRSGHTQ LHGNAIMITS |
| | | | | 421 | TITVVLFSTM VFGMITKPLI RLLLPASGHP RELSEPSSPK SFHSPLLTSQ QGSDLESTTN |
| | | | | 481 | IVRPSSLRGL LTKPTHTVHY YWRKFDDALM RPVFGGRGFV PFVPGSPTER NPPDLSKA |
| 32 | 32396174 | AAP20432 | Na+/H+ | 1 | MSMGYQVVAA QLKVASSADH ASVVIITLFV ALLCACIVLG HLLEENRWLN ESITALIIGL |
| | | | antiporter | 61 | CTGGVILMTT KGKSSHVLVF SEDLFFIYLL PPIIFIAGFQ VKKKQFFRNF MTITLFGAVG |
| | | | NHX5 | 121 | TMISFFTISL GAIAIFSRMN IGTLDVGDFL AIGAIFSATD SVCTLQVLHQ DETPFLYSLV |
| | | | Zea mays | 181 | FGEGVVNDAT SVVLFNAVQK IQITHINAEV ALQVFGNFLY LFSTSTLLGI ATGLITSFVL |
| | | | subsp. | 241 | KKLYFARHST TRELAIMMLM AYLSYMLAEL FSLSGILTVF FCGVLMSHVT WHNVTESSRI |
| | | | mays | 301 | TSRHVFAMLS FIAETFIFLY VGTDALDFDK WKTSSLSFGG TLGVSALIMA LVLLGRAAFV |
| | | | | 361 | FPLSVLTNFS NKHEMESITF KHQVIIWWAG LMRGAVSIAL AFKQFTYSGV TLDPVMAAMV |
| | | | | 421 | TNTTIVVLFT TLVFGLLTKP LIRLLMPHRH LTMLSDDSTP KSLHSPLLTS QLGSDLEEPT |
| | | | | 481 | NIPRPSSIRG EFLTMTRTVH RYWRKFDDAF MRPMFGGRGF VPVVPGSPIE RSVPQWSEEA |
| | | | | 541 | HNKEP |
| 33 | 32396176 | AAP20433 | Na+/H+ | 1 | MGLGVVAELV RLGVLSSTSD HASVVSINLF VALLCACIVL GHLLEENRWV NESITALIIG |
| | | | antiporter | 61 | LCTGVVILLT TKGKSSHILV FSEDLFFIYL LPPIIFNAGF QVKKKQFFRN FMTITLFGAV |
| | | | NHX6 | 121 | GTMISFFTIS LGALGLISRL NIGALELGDY LALGAIFSAT DSVCTLQVLS QDETPFLYSL |
| | | | Zea mays | 181 | VFGEGVVNDA TSVVVFNALQ NFDITHIDAE VVFHLLGNFF YLFLLSTVLG VATGLISALV |
| | | | subsp. | 241 | IKKLYFGRHS TDREVALMML MAYLSYMLAE LFALSGILTV FFGCIVMSHY TWHNVTESSR |
| | | | mays | 301 | ITTKHAFATL SFLAETFLFL YVGMDALDID KWRSVSDTPG KSLAISSILM GLVMVGRAAF |
| | | | | 361 | VFPLSFLSNL AKKTEHEKIS WKQQVVIWWA GLMRGAVSMA LAYKKFTRAG HTQVRGNAIM |
| | | | | 421 | ITSTIIVVLF STMVFGLLTK PLINLLIPHR NATSMLSDDS SPKSLHSPLL TSQLGSDLEE |
| | | | | 481 | PTNIPRPSSI RGEFLTMTRT VHRYWRKFDD AFMRPMFGGR GFVPFVPGSP TERNPPDLSK |
| | | | | 541 | A |
| 34 | 22902099 | AAM54141 | Na+/H+ | 1 | MVAPQLAAVF TKLQTLSTSD HASVVSMNIF VALLCACIVI GHLLEENRWM NESITALIIG |
| | | | antiporter | 61 | VFTGVIILLT SGGKSSHLLV FSEDLFFIYL LPPIIFNAGF QVKKKQFFRN FITIMLFGAV |
| | | | Gossypium | 121 | GTLISCTIIS LGVINFFKEM DIGSLDIGDF LAIGAIFAAT DSVCTLQVLN QDETPLLYSL |
| | | | hirsutum | 181 | VFGEGVVNDA TSVVLFNAIQ SFDLVNTSPR ILLEFIGSFL YLFLASTMLG VIVGLVSAYI |
| | | | | 241 | IKKLYFGRHS TDREFALMML MAYLSYIMAE LFYLSGILTV FFCGIVMSHY TWHNVTESSR |
| | | | | 301 | VTTKHAFATL SFVAETFLFL YVGMDALDME KWRFVSDSPG TSVAVSAVLM GLVMVGRAAF |
| | | | | 361 | VFPLSFLSNL AKKSTSEKIS FREQIIIWWA GLMRGAVSMA LAYNQFTRGG HTQLRGNAIM |
| | | | | 421 | ITSTITIVLF STVVFGLMTK PLIRFLLPHP KPTASMLSDQ STPKSMEAPF LGSGQDSFDD |
| | | | | 481 | SLIGVHRPNS IRALLTTPAH TVHYYWRKFD NAFMRPMFGG RGFVPFVPGS PTERSEPNLP |
| | | | | 541 | QWQ |
| 35 | 30144703 | AAP15178 | Na+/H+ | 1 | MWSQLSSFFA SKMDMVSTSD HASVVSMNLF VALLCGCIVI GHLLEENRWM NESITALLIG |
| | | | antiporter | 61 | LSTGIIILLI SGGKSSHLLV FSEDLFFIYL LPPIIFNAGF QVKKKQFFRN FITIILFGAV |
| | | | Suaeda | 121 | GTLVSFIIIS LGSIAIFQKM DIGSLELGDL LAIGAIFAAT DSVCTLQVLN QDETPLLYSL |
| | | | maritima | 181 | VFGEGVVNDA TSVVLFNAIQ NFDLTHIDHR IAYRIAFQFG GNFLYLFFAS TLLGAVTGLL |
| | | | subsp. | 241 | SAYVIKKLYF GRHSTDREVA LMMLMAYLSY MLAELFYLSG ILTVFFCGIV MSHYTWHNVT |
| | | | salsa | 301 | ESSRVTTKHA FATLSFVAEI FIFLYVGMDA LDIEKWRFVS DSPGTSVAVS SILLGLLMVG |
| | | | | 361 | RALLFSLVFL MNLSKKSNSE KVTFNQQIVI WWAGLMRGAV SVALAYNQFS RSGHTQLRGN |
| | | | | 421 | AIMITSTITV VLFSTMVFGL LTKPLILFML PQPKHFTSAS TVSDLGSPKS FSLPLLEDRQ |
| | | | | 481 | DSEADLGNDD EEAYPRGTIA RPTSLRMLLN APTHTVHHYW RRFDDYFMRP VFGGRGFVPF |
| | | | | 541 | VPGSPTEQST TNLSQRT |
| 36 | 28201131 | BAC56698 | Na+/H+ | 1 | MAFEVVAAQL ARLSDALATS DHASVVSINL FVALLCACIV LGHLLEENRW LNESITALII |
| | | | antiporter | 61 | GLCTGVVILM TTKGKSSHVL VFSEDLFFIY LLPPIIFNAG FQVKKKQFFR NFMTITLFGA |
| | | | Hordeum | 121 | VGTMISFFTI SLAAIAIFSK MNIGTLDVSD FLAIGAIFSA TDSVCTLQVL NQDETPFLYS |
| | | | vulgare | 181 | LVFGEGVVND ATSVVLFNAL QNFDPNQIDA IVILKFLGNF CYLFVSSTFL GVFSGLLSAY |
| | | | | 241 | IIKKLYIGRH STDREVALMM LMAYLSYMLA ELLDLSGILT VFFCGIVMSH YTWHNVTESS |
| | | | | 301 | RVTTKHAFAT LSFIAETFLF LYVGMDALDI EKWKFASDSP GKSIGISSIL LGLVLVGRAA |
| | | | | 361 | FVFPLSFLSN LTKKTELEKI SWRQQIVIWW AGLMRGAVSI ALAYNKFTRS GHTQLHGNAI |
| | | | | 421 | MITSTITVVL FSTMLFGILT KPLIRFLLPA SSNGDPSEPS SPKSLHSPLL TSMLGSDMEA |
| | | | | 481 | PLPIVRPSSL RMLITKPTHT IHYYWRKFDD ALMRPMFGGR GFVPYSPGSP TDPNVIVA |
| 37 | 27948863 | AAO25547 | Na+/H+ | 1 | MGWGLGDPPA DYGSIMAVGL FVALMCICII VGHLLEENRW MNESTTALLL GLGAGTVILF |
| | | | antiporter | 61 | ASSGKNSRLM VFSEDLFFIY LLPPIIFNAG FQVKKKQFFR NFMTITLFAV VGTLISFSII |
| | | | Hordeum | 121 | SLGAMGLISR LNIGALELGD YLALGAIFSA TDSVCTLQVL SQDETPFLYS LVFGEGVVND |
| | | | brevisubu- | 181 | ATSVVLFNAI QNFDLGNFSS LKFLQFIGNF LYLFGASTFL GVASGLLSAY VIKKLYFGRH |
| | | | latum | 241 | STDREVAIMM LMAYLSYMLA ELLDLSGILT VFFCGIVMSH YTWHNVTESS RVTTKHAFAT |
| | | | | 301 | LSFISETFLF LYVGMDALDI EKWKIVSETY SPMKSITLSS IILALVLVAR AAFVFPLSYL |
| | | | | 361 | SNLTKKTAGE KISIRQQVII WWAGLMRGAV SIALAYNKFA KSGHTQLPSN AIMITSTIII |
| | | | | 421 | VLFSTIVFGL LTKPLIRLLI PARHLTREVS ALSEPSSPKS FLEQLTVNGP ETDVENGVSI |
| | | | | 481 | RRPTSLRMLL ASPTRSVHHY WRKFDNAFMR PVFGGRGFVP FVPGSPTESS VPLLAHGSEN |
| 38 | 29825705 | AAO91943 | Vacuolar | 1 | MGPDLGALAL RYTGLAVSDH DSIVAINIFI ALLCGCIVFG HLLEGNRWVN ESTTAIVLGL |
| | | | Na+/H+ | 61 | ITGGVILLCT KGVNSRILIF SEDIFFIYLL PPIIFNAGFQ VKKKQFFRNF ATIILFGAVG |
| | | | antiporter | 121 | TLISFVIITL GAMGLFRKLD VGPLELGDYL AIGAIFSATD SVCTLQVLNQ DQAPLLYSLV |
| | | | Hordeum | 181 | FGEGVVNDAT SVVLFNAIQN IDLNHFDVLV LLQLIGKFLY LFLTSTVLGV AAGLLSAYII |
| | | | vulgare | 241 | KKLCFARHST DREVAIMILM AYLSYMLSML LDLSGILTVF FCGIVMSHYT RHNVTESSRV |
| | | | | 301 | TTKHTFATLS FIAEIFLFLY VGMDALDIDK WKLASSSPKK PIALSAVILG LVMVGRAAFV |
| | | | | 361 | FPLSYLSNLS KKESHPKISF NQQVIIWWAG LMRGAVSIAL AYNKYTTSGH TAVRVNAVMI |
| | | | | 421 | TSTIIVVLFS TMVFGLLTKP LINLLVPPRP GTAADISSQS FLDPLTASLL GSDFDVGQLT |
| | | | | 481 | PQTNLQYLLT MPSRSVHRVW RKFDDKFMRP MFGGRGFVPF VPGSPIERSV HGPGLLGTVT |
| | | | | 541 | EAENRS |

1. A non-naturally occurring salt tolerant plant or plant part from said plant comprising fruit having increased potassium levels when said plant cultivated under elevated salt conditions.

2. The non-naturally occurring salt tolerant plant or plant part from said plant of claim 1 wherein the increased potassium levels are at least 10% higher.

3. The non-naturally occurring salt tolerant plant or plant part from said plant of claim 1 wherein the increased potassium levels are at least 15% higher.

4. The non-naturally occurring salt tolerant plant or plant part from said plant of claim 1 wherein the increased potassium levels are at least 20% higher.

5. The non-naturally occurring salt tolerant plant or plant part from said plant of claim 1 wherein the increased potassium levels are at least 25% higher.

6. The non-naturally occurring salt tolerant plant or plant part from said plant of claim 1 wherein the fruit is a flower developed fruit.

7. The non-naturally occurring salt tolerant plant or plant part from said plant of claim 1 wherein the fruit is an ovary developed fruit.

8. The non-naturally occurring salt tolerant plant or plant part from said plant of claim 1 wherein the plant is transgenic.

9. The non-naturally occurring salt tolerant plant or plant part from said plant of claim 1 wherein the plant is tomato.

10. The non-naturally occurring salt tolerant plant or plant part from said plant of claim 1 wherein the plant is grape.

11. The non-naturally occurring salt tolerant plant or plant part from said plant of claim 1 wherein the plant comprises a transgene.

12. The non-naturally occurring salt tolerant plant or plant part from said plant of claim 11 wherein the transgene comprises a first nucleic acid encoding a Na+/H+ transporter.

13. The non-naturally occurring non-halophyte plant or plant part from said plant of claim 12 wherein the first nucleic acid is selected from the group consisting of the following:
(a) a nucleic acid molecule of the coding strand shown in SEQ ID NO:1, or a complement thereof;
(b) a nucleic acid molecule encoding the amino acid sequence shown in SEQ ID NO:2;
(c) a nucleic acid molecule that hybridizes to the sequence set forth in SEQ ID NO:1 or the complement of the sequence set forth in SEQ ID NO:1 under highly stringent conditions that include at least one wash in 0.1×xSSC, 0.1% SDS, at 65° C. for thirty minutes; and
(d) a nucleic acid molecule encoding a plant NHX transporter polypeptide that hybridizes to the sequence set forth in SEQ ID NO:1 or the complement of the sequence set forth in SEQ ID NO:1 under moderately stringent conditions that includes at least one wash in 0.1×SSC, 0.1% SDS, at 50° C. for thirty minutes.

14. The non-naturally occurring non-halophyte plant or plant part from said plant of claim 13 wherein the transgene further comprises a second nucleic acid operably linked to the first nucleic acid, where in the second nucleic acid comprises a plant promoter.

15. The non-naturally occurring non-halophyte plant or plant part from said plant of claim 14 wherein the promoter is the 35 S promoter.

16. The non-naturally occurring non-halophyte plant or plant part from said plant of claim 14 wherein the plant is tomato.

17. The non-naturally occurring non-halophyte plant or plant part from said plant of claim 1 wherein the plant part is the fruit.

18. A non-naturally occurring non-halophyte seed produced from the plant of claim 1.

19. A transgenic tomato plant comprising a first nucleic acid selected from the group consisting of the following:
(a) a nucleic acid molecule of the coding strand shown in SEQ ID NO:1, or a complement thereof;
(b) a nucleic acid molecule encoding the same amino acid sequence as encoded by the nucleotide sequence of (a);
(c) a nucleic acid molecule that hybridizes to the sequence set forth in SEQ ID NO:1 or the complement of the sequence set forth in SEQ ID NO:1 under highly stringent conditions that include at least one wash in 0.1×SSC, 0.1% SDS, at 65° C. for thirty minutes; and
(d) a nucleic acid molecule encoding a plant NHX transporter polypeptide that hybridizes to the sequence set forth in SEQ ID NO:1 or the complement of the sequence set forth in SEQ ID NO:1 under moderately stringent conditions that includes at least one wash in 0.1×SSC, 0.1% SDS, at 50° C. for thirty minutes.

20. The transgenic tomato plant of claim 19 wherein the transgene further comprises a second nucleic acid operably linked to the first nucleic acid, where in the second nucleic acid comprises a plant promoter.

21. The transgenic tomato plant of claim 20 wherein the promoter is the 35 S promoter.


SEQUENCE LISTING (38 sequences)

SEQ ID NO: 1 - DNA, 2178 bp, Arabidopsis thaliana
cctctctgtttcgttcctcgtagacgaagaagaagaagaatctcaggttttagctttcgaagcttccaaaattttgaattttgatcttctgggctcttttgtaaatcagactgaagatatttagattacccagaagttgttcaaggaatggtttcagtggacagcacggaaagataaaagagacttttttttccagattttgctgatccaaaatctgaatagttgttcatgttcttggatcaaatctggaaagaggaagtttgttggatctagaagaagataacaatgttggattctctagtgtcgaaactgccttcgttatcgacatctgatcacgcttctgtggttgcgttgaatctctttgttgcacttctttgtgcttgtattgttcttggtcatcttttggaagagaatagatggatgaacgaatccatcaccgccttgttgattgggctaggcactggtgttaccattttgttgattagtaaaggaaaaagctcgcatcttctcgtctttagtgaagatcttttcttcatatatcttttgccacccattatattcaatgcagggtttcaagtaaaaaagaagcagtttttccgcaatttcgtgactattatgctttttggtgctgttgggactattatttcttgcacaatcatatctctaggtgtaacacagttctttaagaagttggacattggaacctttgacttgggtgattatcttgctattggtgccatatttgctgcaacagattcagtatgtacactgcaggttctgaatcaagacgagacacctttgctttacagtcttgtattcggagagggtgttgtgaatgatgcaacgtcagttgtggtcttcaacgcgattcagagctttgatctcactcacctaaaccacgaagctgcttttcatcttcttggaaacttcttgtatttgtttctcctaagtaccttgcttggtgctgcaaccggtctgataagtgcgtatgttatcaagaagctatactttggaaggcactcaactgaccgagaggttgcccttatgatgcttatggcgtatctttcttatatgcttgctgagcttttcgacttgagcggtatcctcactgtgtttttctgtggtattgtgatgtcccattacacatggcacaatgtaacggagagctcaagaataacaacaaagcatacctttgcaactttgtcatttcttgcggagacatttattttcttgtatgttggaatggatgccttggacattgacaagtggagatccgtgagtgacacaccgggaacatcgatcgcagtgagctcaatcctaatgggtctggtcatggttggaagagcagcgttcgtctttccgttatcgtttctatctaacttagccaagaagaatcaaagcgagaaaatcaactttaacatgcaggttgtgatttggtggtctggtctcatgagaggtgctgtatctatggctcttgcatacaacaagtttacaagggccgggcacacagatgtacgcgggaatgcaatcatgatcacgagtacgataactgtctgtctttttagcacagtggtgtttggtatgctgaccaaaccactcataagctacctattaccgcaccagaacgccaccacgagcatgttatctgatgacaacaccccaaaatccatacatatccctttgttggaccaagactcgttcattgagccttcagggaaccacaatgtgcctcggcctgacagtatacgtggcttcttgacacggcccactcgaaccgtgcattactactggagacaatttgatgactccttcatgcgacccgtctttggaggtcgtggctttgtaccctttgttccaggttctccaactgagagaaaccctcctgatcttagtaaggcttgagggtaacgtggaagaaaagctttgattttttttggtagaaaagggtgattcaaattatgcttttgtgtaaattatccatttgtaatattgtttgtgaggacagaaatctgtcctaacgttttgagagcagaaagcaaaacatggcaactttgaagtgtttgattgatgtatgtaattatattcatatttgttttgttgtaacacaaactacacatttgtttatgttttgaatttggtttttgcttcgaaaaaaaaaaaaaaaaaaaaaaaaaaaaa

SEQ ID NO: 2 - protein, 538 aa, Arabidopsis thaliana
MLDSLVSKLPSLSTSDHASVVALNLFVALLCACIVLGHLLEENRWMNESITALLIGLGTGVTILLISKGKSSHLLVFSEDLFFIYLLPPIIFNAGFQVKKKQFFRNFVTIMLFGAVGTIISCTIISLGVTQFFKKLDIGTFDLGDYLAIGAIFAATDSVCTLQVLNQDETPLLYSLVFGEGVVNDATSVVVFNAIQSFDLTHLNHEAAFHLLGNFLYLFLLSTLLGAATGLISAYVIKKLYFGRHSTDREVALMMLMAYLSYMLAELFDLSGILTVFFCGIVMSHYTWHNVTESSRITTKHTFATLSFLAETFIFLYVGMDALDIDKWRSVSDTPGTSIAVSSILMGLVMVGRAAFVFPLSFLSNLAKKNQSEKINFNMQVVIWWSGLMRGAVSMALAYNKFTRAGHTDVRGNAIMITSTITVCLFSTVVFGMLTKPLISYLLPHQNATTSMLSDDNTPKSIHIPLLDQDSFIEPSGNHNVPRPDSIRGFLTRPTRTVHYYWRQFDDSFMRPVFGGRGFVPFVPGSPTERNPPDLSKA

SEQ ID NO: 3 - protein, 542 aa, Ipomoea nil
MAFGLSSLLQNSDLFTSDHASVVSMNLFVALLCACIVLGHLLEENRWVNESITALIIGLCTGVVILLLSGGKSSHLLVFSEDLFFIYLLPPIIFNAGFQVKKKQFFVNFMTIMLFGAIGTLISCSIISFGAVKIFKHLDIDFLDFGDYLAIGAIFAATDSVCTLQVLSQDETPLLYSLVFGEGVVNDATSVVLFNAIQSFDMTSFDPKIGLHFIGNFLYLFLSSTFLGVGIGLLCAYIIKKLYFGRHSTDREVALMMLMSYLSYIMAELFYLSGILTVFFCGIVMSHYTWHNVTESSRVTTRHSFATLSFVAETFIFLYVGMDALDIEKWKFVKNSQGLSVAVSSILVGLILVGRAAFVFPLSFLSNLAKKNSSDKISFRQQIIIWWAGLMRGAVSIALAYNKFTTSGHTSLHENAIMITSTVTVVLFSTVVFGLMTKPLINLLLPPHKQMPSGHSSMTTSEPSSPKHFTVPLLDNQPDSESDMITGPEVARPTALRMLLRTPTHTVHRYWRKFDDSFMRPVFGGRGFVPFVAGSPVEQSPR

SEQ ID NO: 4 - protein, 556 aa, Suaeda maritima
MLSQLSSFFASKMDMVSTSDHASVVSMNLFVALLRGCIVIGHLLEENRWMNESITALLIGLSTGIIILLISGGKSSHLLVFSEDLFFIYLLPPIIFNAGFQVKKKQFFRNFITIILFGAVGTLVSFIIISLGSIAIFQKMDIGSLELGDLLAIGAIFAATDSVCTLQVLNQDETPLLYSLVFGEGVVNDATSVVLFNAIQNFDLTHIDHRIAFQFGGNFLYLFFASTLLGAVTGLLSAYVIKKLYFGRHSTDREVALMMLMAYLSYMLAELFYLSGILTVFFCGIVMSHYTWHNVTESSRVTTKHAFATLSFVAEIFIFLYVGMDALDIEKWRFVSDSPGTSVAVSSILLGLHMVGRAAFVFPFAFLMNLSKKSNSEKVTFNQQIVIWWAGLMKSAVSVALAYNQFSRSGHTQLRGNAIMITSTITVVLFSTMVFGLLTKPLILFMLPQPKHFTSASTVSDLGSPKSFSLPLLEDRQDSEADLGNDDEEAYPRGTIARPTSLRMLLNAPTHTVHHYWRRFDDYFMRPVFGGRGFVPFVPGSPTEQSITNFVTENIS

SEQ ID NO: 5 - protein, 540 aa, Petunia x hybrida
MAFDFGTLLGNVDRLSTSDHQSVVSINLFVALICACIVIGHLLEENRWMNESITALVIGSCTGIVILLISGGKNSHILVFSEDLFFIYLLPPIIFNAGFQVKKKSFFRNFSTIMLFGALGTLISFIIISLGAIGIFKKMNIGSLEIGDYLAIGAIFSATDSVCTLQVLNQDETPLLYSLVFGEGVVNDATSVVLFNAIQNFDLSHIDTGKAMELVGNFLYLFASSTALGVAAGLLSAYIIKKLYFGRHSTDREVAIMILMAYLSYMLAELFYLSAILTVFFSGIVMSHYTWHNVTESSRVTTKHTFATLSFIAEIFIFLYVGMDALDIEKWKFVSDSPGISVQVSSILLGLVLVGRAAFVFPLSFLSNLTKKTPEAKISFNQQVTIWWAGLMRGAVSMALAYNQFTRGGHTQLRANAIMITSTITVVLFSTVVFGLMTKPLIRILLPSHKHLSRMISSEPTTPKSFIVPLLDSTQDSEADLERHVPRPHSLRMLLSTPSHTVHYYWRKFDNAFMRPVFGGRGFVPFAPGSPTDPVGGNLQ

SEQ ID NO: 6 - protein, 555 aa, Torenia hybrida
MGFESVIKLAASETDNLWSSGHGSVVAITLFVTLLCTCIVIGHLLEENRWMNESIIALIIGLATGVIILLISGGKSSHLLVFSEDLFFIYALPPIIFNAGFQVKKKSFFRNFATIMMFGAVGTLISFIIISLGTIAFFPKMNMRLGVGDYLAIGAIFAATDSVCTLQVLSQDETPLLYSLVFGEGVVNDATSVVLFNAVQNFDLPHMSTAKAFELVGNFFYLFATSTVLGVLTGLLSAYIIKKLYFGRHSTDREVAIMILMAYLSYMLAELFDLSGILTVFFCGIVMSHYTWHNVTENSRVTTKHTFATLSFVAEIFIFLYVGMDALDIEKWRFVSGSMTTSAAVSATLLGLVLLSRAAFVFPLSFLSNLAKKSPLEKISLRQQIIIWWAGLMRGAVSMALAYKQFTREGLTVERENAIFITSTITIVLFSTVVFGLMTKPLINLLIPSPKLNRSVSSEPLTPNSITIPLLGESQDSVAELFSIRGQTSQGGEPVARPSSLRMLLTKPTHTVHYYWRKFDNAFMRPVFGGRGFVPYVPGSPTERSVRNWEEETKQ

SEQ ID NO: 7 - protein, 542 aa, Ipomoea tricolor
MAFGLSSLLQNSELFTSDHASVVSMNLFVALLCACIVLGHLLEENRWVNESITALIIGLCTGVVILLLSRGKSSHLLVFSEDLFFIYLLPPIIFNAGFQVKKKQFFVNFMTIMLFGAIGTLISCSIISFGAVKIFKHLDIDFLDFGDYLAIGAIFAATDSVCTLQVLSQDETPLLYSLVFGEGVVNDATSVVLFNAIQSFDMTSFDPKIGLHFIGNFLYLFLSSTFLGVGIGLLCAYIIKKLYFGRHSTDREVALMMLMSYLSYIMAELFYLSGILTVFFCGIVMSHYTWHNVTESSRVTTRHSFATLSFVAETFIFLYVGMDALDIEKWKFVKNSQGLSVAVSSILVGLILVGRAAFVFPLSFLSNLAKKNSSDKISFRQQIIIWWAGLMRGAVSIALAYNKFTTSGHTSLHENAIMITSTVTVVLFSTVVFGLMTKPLINLLLPPHKQIASGHSSMTTSEPSSPKHFAVPLLDNQHDSESDMITGPEVARPTALRMLLRTPTHTVHRYWRKFDDSFMRPVFGGRGFVPFVAGSPAEQSPR

SEQ ID NO: 8 - protein, 575 aa, Arabidopsis thaliana
MISPVEHDPQGQVKQQQAAGVGILLQIMMLVLSFVLGHVLRRHRFHYLPEASGSLLIGLIVGILANISDTETSIRFCPPPSIPEFSLLSFPRSLVCSFYSVSGRGLISTKSSSSCFCCLPSYYILCFNICISSFKFAAAMLCIMDVIFLDIIHLFEPSQVSVFNLNHSFLTLEPLLPLLSSELLSLQLLLVVCYLGGSMYLMYKLPFVECLMFGALISATDPVTVLSIFQVLLLFLLLSVSTGYKYSHDVGTDVNLYALVFGESVLNDAVSFYYLLRYWALPFKTMSLVNRQSSSGEHFFMVVIRFFETFAGSMSAGLAISFLNSFYTVVFTLLILSEHIVNVMSLFSLFSTSIHACRRCWSLRHCFYTLHRNCNRRVMKRYTFSNLSEASQSFVSSFFHLISSLAETFTFIYMGFDIAMEQHSWSHVGAVNVFGCAYLVNLFRQENQKIPMKHQKALWYSGLRGAMAFALALQSLHDLPEGHGQIIFTATTTIVVVTVTFVLLIGGSTGKMLEALEVVGDDLDDSMSEVNSRRSTLISLNIGASSDEDTSSSGSRFKMKLKEFHKTGDGDGDGE

SEQ ID NO: 9 - protein, 1146 aa, Arabidopsis thaliana
MTTVIDATMAYRFLEEATDSSSSSSSSKLESSPVDAVLFVGMSLVLGIASRHLLRGTRVPYTVALLVIGIALGSLEYGAKHNLGKIGHGIRIWNEIDPELLLAVFLPALLFESSFSMEVHQIKRCLGQMVLLAVPGVLISTACLGSLVKVTFPYEWDWKTSLLLGGLLSATDPVAVVALLKELGASKKLSTIIEGESLMNDGTAIVVFQLFLKMAMGQNSDWSSIIKFLLKVALGAVGIGLAFGIASVIWLKFIFNDTVIEITLTIAVSYFAYYTAQEWAGASGVLTVMTLGMFYAAFARTAFKGDSQKSLHHFWEMVAYIANTLIFILSGVVIAEGILDSDKIAYQGNSWRFLFLLYVYIQLSRVVVVGVLYPLLCRFGYGLDWKESIILVWSGLRGAVALALSLSVKQSSGNSHISKETGTLFLFFTGGIVFLTLIVNGSTTQFVLRLLRMDILPAPKKRILEYTKYEMLNKALRAFQDLGDDEELGPADWPTVESYISSLKGSEGELVHHPHNGSKIGSLDPKSLKDIRMRFLNGVQATYWEMLDEGRISEVTANILMQSVDEALDQVSTTLCDWRGLKPHVNFPNYYNFLHSKVVPRKLVTYFAVERLESACYISAAFLRAHTIARQQLYDFLGESNIGSIVINESEKEGEEAKKFLEKVRSSFPQVLRVVKTKQVTYSVLNHLLGYIENLEKVGLLEEKEIAHLHDAVQTGLKKLLRNPPIVKLPKLSDMITSHPLSVALPPAFCEPLKHSKKEPMKLRGVTLYKEGSKPTGVWLIFDGIVKWKSKILSNNHSLHPTFSHGSTLGLYEVLTGKPYLCDLITDSMVLCFFIDSEKILSLQSDSTIDDFLWQESALVLLKLLRPQIFESVAMQELRALVSTESSKLTTYVTGESIEIDCNSIGLLLEGFVKPVGIKEELISSPAALSPSNGNQSFHNSSEASGIMRVSFSQQATQYIVETRARAIIFNIGAFGADRTLHRRPSSLTPPRSSSSDQLQRSFRKEHRGLMSWPENIYAKQQQEINKTTLSLSERAMQLSIFGSMVNVYRRSVSFGGIYNNKLQDNLLYKKLPLNPAQGLVSAKSESSIVTKKQLETRKHACQLPLKGESSTRQNTMVESSDEEDEDEGIVVRIDSPSKIVFRNDL

SEQ ID NO: 10 - protein, 555 aa, Atriplex gmelini
MWSQLSSLLSGKMDALTTSDHASVVSMNLFVALLCGCIVIGHLLEENRWMNESITALLIGLATGVVILLISGGKSSHLLVFSEDLFFIYLLPPIIFNAGFQVKKKQFFRNFITIVLFGAVGTLVSFTIISLGALSIFKKLDIGTLELADYLAIGAIFAATDSVCTLQVLNQDETPLLYSLVFGEGVVNDATSVVLFNAIQSFDLTRIDHRIALQFMGNFLYLFIASTILGAFTGLLSAYIIKKLYFGRHSTDREVALMMLMAYLSYMLAELFYLSGILTVFFCGIVMSHYTWHNVTESSRVTTKHAFATLSFVAEVFLFLYVGMDALDIEKWRFVSDSPGISVAVSSILLGLVMVGRAAFVFPLSWLMNFAKKSQSEKVTFNQQIVIWWAGLMRGAVSMALAYNQFTRSGHTQLRGNAIMITSTISVVLFSTMVFGLLTKPLIMFLLPQPKHFTSCSTVSDVGSPKSYSLPLLEGNQDYEVDVGNGNHEDTTEPRTIVRPSSLRMLLNAPTHTVHHYWRKFDDSFMRPVFGGRGFVPFVPGSPTEQSTNNLVDRT

SEQ ID NO: 11 - protein, 985 aa, Saccharomyces cerevisiae
MAIWEQLEVSKAHVAYACVGVFSSIFSLVSLYVKEKLYIGESTVAGIFGLIVGPVCLNWFNPLKWGNSDSITLEITRIVLCLQIFAVAVELPRKYMLKHWVSVTMLLLPVMTAGWLIIGLFVWILIPGLNFSASLLISACITATDPILAQSVVSGKFAQRVPGHLRNLLSAESGCNDGMAFPFLFLSMNLILHPGNGREIVKDWICVTILYECLFGCLLGCFIGYVGRITIRFAEKKNIIDRESFLAFYVVLAFMCAGFGSILGVDDLLVSFAAGATFAWDGWFSQKTQESNVSTVIDLLLNYAYFIYFGAIIPWSQFNNGEIGTNVWRLIILSIVVIFLRRIPAVMILRPLIPDIKSWREALFVGHFGPIGVGAIFAAILARGELESTFSDEPTPLNVVPSKEESKHWQLIACIWPITCFFIVTSIIVHGSSVAIITLGRHLNTITLTKTFTTHTTNGDNGKSSWMQRLPSLDKAGRSFSLHRMDTQMTLSGDEGEAEEGGGRKGLAGGEDEEGLNNDQIGSVATSGIPARPAGGMPRRRKLSRKEKRLNRRQKLRNKGREIFSSRSKNEMYDDDELNDLGRERLQKEKEARAATFALSTAVNTQRNEEIGMGGDEEEDEYTPEKEYSDNYNNTPSFESSERSSSLRGRTYVPRNRYDGEETESEIESEDEMENESERSMASSEERRIRKMKEEEMKPGTAYLDGNRMIIENKQGEILNQVDIEDRNEARDDEVSVDSTAHSSLTTTMTNLSSSSGGRLKRILTPTSLGKIHSLVDKGKDKNKNSKYHAFKIDNLLIIENEDGDVIKRYKINPHKSDDDKSKNRPRNDSVVSRALTAVGLKSKANSGVPPPVDEEKAIEGPSRKGPGMLKKRTLTPAPPRGVQDSLDLEDEPSSEEDLGDSYNMDDSEDYDDNAYESETEFERQRRLNALGEMTAPADQDDEELPPLPVEAQTGNDGPGTAEGKKKQKSAAVKSALSKTLGLNK

SEQ ID NO: 12 - protein, 633 aa, Saccharomyces cerevisiae
MLSKVLLNIAFKVLLTTAKRAVDPDDDDELLPSPDLPGSDDPIAGDPDVDLNPVTEEMFSSWALFIMLLLLISALWSSYYLTQKRIRAVHETVLSIFYGMVIGLIIRMSPGHYIQDTVTFNSSYFFNVLLPPIILNSGYELNQVNFFNNMLSILIFAIPGTFISAVVIGIILYIWTFLGLESIDISFADAMSVGATLSATDPVTILSIFNAYKVDPKLYTIIFGESLLNDAISIVMFETCQKFHGQPATFSSVFEGAGLFLMTFSVSLLIGVLIGILVALLLKHTHIRRYPQIESCLILLIAYESYFFSNGCHMSGIVSLLFCGITLKHYAYYNMSRRSQITIKYIFQLLARLSENFIFIYLGLELFTEVELVYKPLLIIVAAISICVARWCAVFPLSQFVNWIYRVKTIRSMSGITGENISVPDEIPYNYQMMTFWAGLRGAVGVALALGIQGEYKFTLLATVLVVVVLTVIIFGGTTAGMLEVLNIKTGCISEEDTSDDEFDIEAPRAINLLNGSSIQTDLGPYSDNNSPDISIDQFAVSSNKNLPNNISTTGGNTFGGLNETENTSPNPARSSMDKRNLRDKLGTIFNSDSQWFQNFDEQVLKPVFLDNVSPSLQDSATQSPADFSSQNH

SEQ ID NO: 13 - protein, 546 aa, Arabidopsis thaliana
MTMFASLTSKMLSVSTSDHASVVSLNLFVALLCACIVIGHLLEENRWMNESITALLIGLGTGVVILLISRGKNSHLLVFSEDLFFIYLLPPIIFNAGFQVKKKQFFRNFVTIMAFGAIGTVVSCTIISLGAIQFFKKLDIGTFDLGDFLAIGAIFAATDSVCTLQVLNQDETPLLYSLVFGEGVVNDATSVVLFNAIQSFDLTHLNHEAAFQFLGNFFYLFLLSTGLGVATGLISAYVIKKLYFGRHSTDREVALMMLMAYLSYMLAELFALSGILTVFFCGIVMSHYTWHNVTESSRITTKHAFATLSFLAETFIFLYVGMDALDIEKWRFVSDSPGTSVAVSSILMGLVMLGRAAFVFPLSFLSNLAKKHQSEKISIKQQVVIWWAGLMRGAVSMALAYNKFTRSGHTELRGNAIMITSTITVCLFSTMVFGMLTKPLIRYLMPHQKATTSTTSMLSDDSTPKSIHIPLLDGEQLDSFELPGSHQDVPRPNSLRGFLMRPTRTVHYYWRQFDDAFMRPVFGGRGFVPFVPGSPTERSSHDLSKP

SEQ ID NO: 14 - protein, 529 aa, Arabidopsis thaliana
MSIGLTEFVTNKLAAEHPQVIPISVFIAILCLCLVIGHLLEENRWVNESITAILVGAASGTVILLISKGKSSHILVFDEELFFIYLLPPIIFNAGFQVKKKKFFHNFLTIMSFGVIGVFISTVIISFGTWWLFPKLGFKGLSARDYLAIGTIFSSTDTVCTLQILHQDETPLLYSLVFGEGVVNDATSVVLFNAVQKIQFESLTGWTALQVFGNFLYLFSTSTLLGIGVGLITSFVLKTLYFGRHSTTRELAIMVLMAYLSYMLAELFSLSGILTVFFCGVLMSHYASYNVTESSRITSRHVFAMLSFIAETFIFLYVGTDALDFTKWKTSSLSFGGTLGVSGVITALVLLGRAAFVFPLSVLTNFMNRHTERNESITFKHQVIIWWAGLMRGAVSIALAFKQFTYSGVTLDPVNAAMVTNTTIVVLFTTLVFGFLTKPLVNYLLPQDASHNTGNRGKRTEPGSPKEDATLPLLSFDESASTNFNRAKDSISLLMEQPVYTIHRYWRKFDDTYMRPIFGGPRRENQPEC

SEQ ID NO: 15 - protein, 552 aa, Arabidopsis thaliana
MVIGLSTMLEKTEALFASDHASVVSMNLFVALLCACIVLGHLLEETRWMNESITALIIGSCTGIVILLISGGKSSRILVFSEDLFFIYLLPPIIFNAGFQVKKKQFFRNFMTIMLFGAIGTLISFVIISFGAKHLFEKMNIGDLTIADYLAIGAIFSATDSVCTLQVLNQDETPLLYSLVFGEGVVNDATSVVLFNAIQRFDLTNINSAIALEFAGNFFYLFILSTALGVAAGLLSAFVIKKLYIGRHSTDREVALMMLLAYLSYMLAELFHLSSILTVFFCGIVMSHYTWHNVTDKSKVTTKHTFAAMSFLAEIFIFLYVGMDALDIEKWDVVRNSPGQSIGVSSILLGLILLGRAAFVFPLSFLSNLTKSSPDEKIDLKKQVTIWWAGLMRGAVSMALAYNQFTTSGHTKVLGNAIMITSTITVVLFSTVVFGLLTKPLVKHLQPSSKQSSTTALQITLRSSFHDPILHEPLLSTQGQSEYDPEQHVSFRMFWKSPSRAIHHYWRKFDNAVMRRIFGGRGVSPVVPGSPIENSVPQWSEEVENKEQNGEP

SEQ ID NO: 16 - protein, 487 aa, Arabidopsis thaliana
MEEVMISPVEHDPQGQVKQQQAAGVGILLQIMMLVLSFVLGHVLRRHRFHYLPEASGLIVGILANISDTETSIRFCPPPSIPEFSLLSFPRSLKPFFSNFGAIVTFAIIGTFVASVVTGGLVYLGGSMYLMYKLPFVECLMFGALISATDPVTVLSIFQDVGTDVNLYALVFGESVLNDAVSFYYLLRYWALPFKFFETFAGSMSAEHLFKYAGLDTENLQNLECCLFVLFPYFSYMLAEGVGLSGIVSILFTGIVMKRYTFSNLSEASQSFVSSFFHLISSLAETFTFIYMGFDIAMEQHSWSHVGFILFSIVSSFTDRQAVNVFGCAYLVNLFRQENQKIPMKHQKALWYSGLRGAMAFALALQSLHDLPEGHGQIIFTATTTIVVVTVLLIGGSTGKMLEALEVVGDDLDDSMSEGFEESDHQYVPPPFSIGASSDEDTSSSGSRFKMKLKEFHKTTTSFTALDKNFLTPFFTTNSGDGDGDGE

SEQ ID NO: 17 - protein, 531 aa, Arabidopsis thaliana
MSSELQISPAIHDPQGQEKQQQAAGVGILLQIMMLVLSFVLGHVLRRHKFYYLPEASASLLIGLIVGGLANISNTETSIRFVELFLISFFRHGSISTMSSSFCFCCLPSYYILKIEYLGGVMFLMYRLPFVECLMFGSLISATDPVTVLSIFQELGSDVNLYALVFGESVLNDADEIVTLLIRSFSFLCCFWQMAISLYRTMSLVRSHSSGQNFFMVIVRFLETFVGSMSAAMKYFILMYSLLLSVYRTWSAVSSYFFHISRNKTLLFYTSYVSIYFTLIEIVQFVMKHYTYSNLSANSQRFVSAFFHLISSLAETFVFIYMGFDIAMEKHSWAANVFGCGYLVNLARPAHRKIPMTHQKALWYSGKILLCVPLSSYCFYSSVINTKICGFCIGLRGAMAFALALQSVHDLPEGHGQTIFTATTAIVVLTVLLIGGSTGTMLEALEVVGDSHDTSLGDGFEVVNSRYMTSYDDEDTPPGSGFRTKLREFHKSAASFTELDRNYLTPFFTSNNGDYDDEGNMEQHHGNNIIL

SEQ ID NO: 18 - protein, 697 aa, Arabidopsis thaliana
MTSIIGAALPYKSPEKAIASSSYSAENDSSPVDAVIFAGTSLVLGTACRYLFNGTRVPYTVVLLVIGIFLGSLEYGTKHNLGKLGHGIRIWNGINPDLLLAVFLPVLLFESSFSMDVHQIKRCMGQMVLLAGPGVLISTFCLGALIKLTFPYNWDWKTSLLLGGLLGATDPVAVVALLKELGASKKMTTLIDGESLMNDGVSVVVFQLFFKMVMGHNSDWGSIIKFLVQNSFGAVGIGLAFGIASVFWLKFIFNDTVAQITVTLSASYFAYYTAQEWAGVSGILTVMILGMFFAAFARTAFKGDSHQSLHHFWYFTTQEMAAYIANTLVFMLSGVIIAESVLSGQTISYKAIKWKFISQFRYGNKAVLQFLFLTGGIVFLTLVVNGSTTQLLLHLLRMDTLTATKKRILEYTKFEMMKTALKAFENLGDDEELGSADWPTVIRHISSLKDLEGRQVNPHDGYEAGSLDPTNIMDIRVQAAYWEMLDDGRITQCTANVLMQSVDEALDLVSTSSLSDWRGLEPRVHFPNYYKFLQSKIIPHKLVTHLIVERLESACYISSAFLRAHRIARQQLHIFLGNSNIASTVINESEVEGEEAKQFLEDVRDSFPQVLSVLKTRQVTHYVLNHLNGYIKNLEKVGLLEGKEVSHLHDVVQSDLKKLLRHPPSLKLPNVDDLITSNPLLKDRSSFRSLAIGETDA

SEQ ID NO: 19 - protein, 697 aa, Arabidopsis thaliana; identical to SEQ ID NO: 18

SEQ ID NO: 20 - protein, 534 aa, Lycopersicon esculentum
MGLDAVARLGVSILSDGDQVSVDSITLFVALLCGCIVIGHLLEESRWINDSITTLVIGLSTGGIILLTTKGKSSHLLEFDEQLFFIYVLPPIIFNAGFQVKKKQFFRNFVTIMLFGAVGTLISFSIISFGAKELLGKLDIGFLELRDYLAIGAIFSATDSVCTLQALNQDETPRLYSLVFGEGVVNDATSVVLFNAIQKLDLSHINSRAALVFTGNFLYLFLASTFLGVLIGLLSAYLIKKIYLGRHSTDREVALMILMAYLSYVMAELFDLSGILTVFICGIVMSHYTWHNVTFNSKVTTRHAFATLSFIAEIFIFLYVGMDALDIEKWRFVKDSPGKSVGVSAALLGLVLVGRACFVFPLSLFSNCLKRSEHDKFGLKLQVTIWWAGLMRGSVSMALAYNQFTRFGHTQQPGNAVMITSTITIVLFSTVVFGLITKPLVRFLLPSSQGFNNLISSEQSFARPLLTNEQELELEMGNVDPVRPSGLSILLKEPSYTIHNHWRRFDDAFMRPLFGGRGFVPDAPELSKGGCDQY

SEQ ID NO: 21 - protein, 531 aa, Lycopersicon esculentum
MEDHLQISPAGAKAIPGKEQQAAGYGILLQIMMLVLSFVIGHVLRRRHFYYIPEASASLLIGLIVGGLANVSDTETSIRAWFNFHEEFFFLFLLPPIIFQSGFSLSPKPFFSNFGAIITFAILGTFIASFVTGILVYLGGVTYLMYRLPFVECLMFGALISATDPVTVLSIFQELGTDVNLYALVFGESVLNDAMAISLYRTMSLVRSHMSTDQNYFMITIRFVETFMGSLSAGVGVGFVSALLFKYAGLDIDNLQNLESCLFVLFPYFSYMLAEGLGLSGIVSILFTGVVMKRYTYPNLSESSQRFVSAFFHLISSLAETFVFIYMGFDIAMEKHSWSHVGFIFFSILFIVIARAANVFGCAYLVNLVRPPHQKIPAKHQKALWYSGLRGAMAFALALQPVHDLPEGHGQAIFTATTAIVVLTVLIIGGSAGTMLEALEVVGDGQSGSMDETFEGNNGYIAPSYRDESYDGEPSSGNRFRMKLKEFHKSTTSFSALDKNYLTPFFTTQGGDEDEDEPIMHSSRRAGYDGH

SEQ ID NO: 22 - protein, 535 aa, Oryza sativa
MGMEVAAARLGALYTTSDYASVVSINLFVALLCACIVLGHLLEENRWVNESITALIIGLCTGVVILLMTKGKSSHLFVFSEDLFFIYLLPPIIFNAGFQVKKKQFFRNFMTITLFGAVGTMISFFTISIAAIAIFSRMNIGTLDVGDFLAIGAIFSATDSVCTLQVLNQDETPFLYSLVFGEGVVNDATSIVLFNALQNFDLVHIDAAVVLKFLGNFFYLFLSSTFLGVFAGLLSAYIIKKLYIGRHSTDREVALMMLMAYLSYMLAELLDLSGILTVFFCGIVMSHYTWHNVTESSRVTTKHAFATLSFIAETFLFLYVGMDALDIEKWEFASDRPGKSIGISSILLGLVLIGRAAFVFPLSFLSNLTKKAPNEKITWRQQVVIWWAGLMRGAVSIALAYNKFTRSGHTQLHGNAIMITSTITVVLFSTMVFGMMTKPLIRLLLPASGHPVTSEPSSPKSLHSPLLTSMQGSDLESTTNIVRPSSLRMLLTKPTHTVHYYWRKFDDALMRPMFGGRGFVPFSPGSPTEQSHGGR

SEQ ID NO: 23 - protein, 553 aa, Nierembergia caerulea
MAFDFGTLLGKMNNLTTSDHQSVVSVNLFVALICACIVIGHLLEENRWMNESITALVIGSCTGVIILLISGGKNSHILVFSEDLFFIYLLPPIIFNAGFQVKKKSFFRNFSTIMLFGAVGTLISFIIISAGAIGIFKKMDIGHLEIGDYLAIGAIFAATDSVCTLQVLNQEETPLLYSLVFGEGVVNDATSVVLFNAVQNFDLSHISTGKALQLIGNFLYLFASSTFLGVAVGLLSAFIIKKLYFGRHSTDREVAIMILMAYLSYMLAELFYLSGILTVFFCGIVMSHYTWHNVTESSRVTTKHTFATLSFIAEIFIFLYVGMDALDIEKWKFVSDSPGTSIKVSSILLGLVLVGRGAFVFPLSFLSNLTKKNPEDKISFNQQVTIWWAGLMRGAVSMALAYNQFTRGGHTQLRANAIMITSTITVVLFSTVVFGLMTKPLILLLLPSQKHLIRMISSEPMTPKSFIVPLLDSTQDSEADLGRHVPRPHSLRMLLSTPSHTVHYYWRKFDNAFMRPVFGGRGFVPFVPGSPTEPVEPTEPRPAESRPTEPTDE

SEQ ID NO: 24 - protein, 542 aa, Citrus x paradisi
MDQAISSVVRKLQMVNTSDHNSVVSINIFVALPCASIVIGHLLEESRWMNESITALLIGVCAGVIILLTTGGKSSHLFVFSEDLFFIYVLPPIIFNAGFQVKKKQFFRNFITIMLFGAIGTLVSCTIISLGVIQFFKKLDIGTLDIGDYLAIGAIFAATDSVCTLQVLNQDDTPLLYSLVFGEGVVNDATSVVLFNAIQSFDLTHINTRSAFQFIGNFLYLFFTSTLLGVIGGLLSAYVIKKLYFGRHSTDREVAIMVLMAYLSYMLAELFYLSGILTVFFCGIVMSHYTWHNVTESSRVTTKHTFATLSFVAEIFTFLYVGMDALDIEKWRFVKGSPGTSVAASAMLMGLIMAGRAAFVFPLSFLTNLAKKSPTEKISIKQQVIIWWAGLMRGAVSMALAYNQFTRSGHTQLRENAIMITSTITVVLFSTVVFGLMTEPLIRLLLPHPKHTTNHILSDPSTPKSLSQPLLEEGQQDSYADLVGPTVPRPGSLRALLTTPTHTVHYYWRKFDDAFMRPVFGGRGFAPFVPGSPTERSVRGGQ

SEQ ID NO: 25 - protein, 546 aa, Triticum aestivum
MGLDLGALALKYTGLAVSDHDSIVAINIFIALLCGCIVFGHLLEGNRWVNESTTALVLGLITGGVILICTKGVNSRILIFSEDIFFIYLLPPIIFNAGFQVKKKQFFRNFATIILFGAAGTLISFVIITFGAMGLFSKLDVGPLELGDYLAIGAIFSATDSVCTLQVLNQDEAPLLYSLVFGEGVVNDATSVVLFNAIQNIDINHFDVFVLLQFIGKFLYLFFTSTVLGVAAGLLSAYIIKKLCFARHSTDREVAIMILMAYLSYMLSMLLDLSGILTVFFCGIVMSHYTWHNVTESSRVTTKHTFATLSFIAEIFLFLYVGMDALDIDKWKLASSSPKKPIALSAVILGLVMVGRAAFVFPLSFLSNLSKKESHPKISFNQQVIIWWAGLMRGAVSIALAYNKFTTSGHTAVRVNAVMITSTIIVVLFSTMVFGLLTKPLINLLIPPRPGTAADISSQSFLDPLTASLLGSDFDVGQLTPQTNLQYLLTMPTRSVHRVWRKFDDKFMRPMFGGRGFVPFVPGSPIERSVHGPGLLGTVTEAEDRS

SEQ ID NO: 26 - protein, 538 aa, Triticum aestivum
MGYQVVAAQLARLSGALGTSDHASVVSITLFVALLCACIVLGHLLEENRWLNESITALIIGLCTGVVILMTTKGKSSHVLVFSEDLFFIYLLPPIIFNAGFQVKKKQFFRNFMAITLFGAVGTMMSFFTISLAAIAIFSRMNIGTLDVSDFLAIGAIFSATDSVCTLQVLNQDETPFLYSLVFGEGVVNDATSVVLFNALQNFDPNQIDAIVILKFLGNFCYLFVSSTFLGVFTGLLSAYVIKKLYIGRHSTDREVALVMLMAYLSYMLAELLDLSGILTVFFCGIVMSHYTWHNVTESSRVTTKHAFATLSFIAETFLFLYVGMDALDIEKWKFASDSPGKSIGISSILLGLVLVGRAAFVFPLSFLSNLTKKTELEKISWRQQIVIWWAGLMRGAVSIALAYNKFTRSGHTQLHGNAIMITSTITVVLFSTMLFGILTKPLIRFLLPASSNGAASDPASPKSLHSPLLTSQLGSDLEAPLPIVRPSSLRMLITKPTHTIHYYWRKFDDALMRPMFGGRGFVPYSPGSPTDPNVLVE

SEQ ID NO: 27 - protein, 546 aa, Triticum aestivum
MGLDLGALALKYTGLAVSDHDSIVAINIFIALLCGCIVFGHLLGGNRWVNESTAALVLGLITGGVILICTKGVNSRILIFSEDIFFIYLLPPIIFNAGFQVKKKQFFRNFATIILFGAAGTLISFVIITFGAMGLFSKLDVGPLELGDYLAIGAIFSATDSVCTLQVLNQDEAPLLYSLVFGEGVVNDATSVVLFNAIQNIDINHFDVFGLLQFIGKFLYLFFTSTVLGVAAGLLSAYIIKKLCFARHSTDREVAIMILMAYLSCMLSMLLDLSGILTVFFCGIVMSHYTWHNVTESSRVTTKHTFATLSFIAEIFLFLYVGMDALDIDKWKLASSSPKKPIALSAVILGLVMVGRAAFVFPLSFLSNLSKKESHPKISFNQQVIIWWAGLMRGAVSIALAYNKFTTSGHTAVRVNAVMITSTIIVVLFSTMVFGLLTKPLINLLIPPRPGTAADISSQSFLDPLTASLLGSDFDVGQLTPQTNLQYLLTMPTRSAHRVWRKFDDKFMRPMFGGRGFVPFVPGSPIERSVHGPGLLGTVTEAEDRS

SEQ ID NO: 28 - protein, 540 aa, Zea mays
MGLGVVAELVRLGVLSSTSDHASVVSINLFVALLCACIVLGHLLEENRWVNESTALIVGLGTGTVILMISRGVVIHVLVFSEDLFFFYLLPPIIFNAGFQVKKKQFFRNFITITLFGAVGTLISFTVISLGALGLISRLNIGALELGDYLALGAIFSATDSVCTLQVLSQDETPFLYSLVFGEGVVNDATSVVVFNALQNFDITHIDAEVVFHLLGNFFYLFLLSTVLGVATGLISALVIKKLYFGRHSTDREVALMMLMAYLSYMLAELFALSGILTVFFGCIVMSHYTWHNVTESSRITTKHAFATLSFLAETFLFLYVGMDALDIDKWRSVSDTPGKSLAISSILMGLVMVGRAAFVFPLSFLSNLAKKTEHEKISWKQQVVIWWAGLMRGAVSMALAYKKFTRAGHTQVRGNAIMITSTIIVVLFSTMVFGLLTKPLINLLIPHRNATSMLSDDSSPKSLHSPLLTSQLGSDLEEPTNIPRPSSIRGEFLTMTRTVHRYWRKFDDAFMRPMFGGRGFVPFVPGSPTERNPPDLSKA

SEQ ID NO: 29 - protein, 540 aa, Zea mays
MGLGVDAETVRLGVLSSTSDHASVVSNNFFVALLCACIVLGHLLEENRMVNESITALLVGLGTGTVILMISRGVSIHVLVFSEDLFFIYLLPPIIFNAGFQVKKKQFFRNFITIILFGAIGTLISFVIISLGAMGLFKKLDVGPLELGDYLAIGAIFSATDSVCTLQVLNQDETPLLYSLVFGEGVVNDATSIVVFNALQNFDITHINAEVVFHLLGNFLYLFLLSTVLGVATGLISALVIKKIYFGRHSTDREVALMMLMAYLSYMLAELFALSGILTVFFGCIVMSHYTWHNVTESSRITTKHAFATLSFLAETFIFLYVGMDALDIEKWRSVSDTPGKSIAISSILMGLVMLGRAAFVFPLSFLSNLAKKNEHEKISWKQQVVIWWSGLMRGAVSMALAYNKFTRAGHTEVRGNEIMITSTITVVLFSTVVFGLLTKPLIRLLMPHRHLTMLSDDSTPKSLHSPLLTSQLGSSIEEPTQIPRPTNIRGEFTTMTRTVHRYWRKFDDKFMRPMFGGRGFVPFVPGSPTERNPHDLSKP

SEQ ID NO: 30 - protein, 539 aa, Zea mays
MSIGLTAETVTNKLASAEHPQVVPNSVFIALLCLCLVIGHLLEENRWVNESITAILVGAATGTVILLISKGKSSHILVFDEELFFIYLLPPIIFNAGFQVKKKQFFRNFITIILFGAIGTLISFVIISLGAMGLFKKLDVGPLELGDYLAIGAIFSATDSVCTLQVLNQDETPLLYSLVFGEGVVNDATSVVLFNAVQKIDFEHLTGEVALQVFGNFLYLFSTSTVLGIATGLITAFVLKTLYFGRHSTTRELAIMVLMAYLSFMLAELFSLSGIITVFFCGVLMSHVTWHNVTESSRITSRHVFAMLSFIAETFLFLYVGTDALDFTKWKTSSLSFGKSLGVSSVLLGLVLVGRAAFVFPLSFLSNLSKKHPGEKITIRQQVVIWWAGLMRGAVSIALAFNKFTRAGHTQVRGNAIMITSTIIVVLFSTVVFGLLTKPLINLLIPHRNATSMLSDDSSPKSLHSPLLTSQLISSIEEPTQIPRPTNIRGEFMTMTRTVHRYWRKFDDKFMRPMFGGRGFVPFVPGSPTERSSPDLSKA

SEQ ID NO: 31 - protein, 538 aa, Zea mays
MGYQVVAAQLKLASSADHASVVIITLFVALLCACIVLGHLLEENRWLNESITALIIGLGTGVVILLISRGKNSRLLVFSEDLFFIYLLPPIIFNAGFQVKKKQFFRNFMTITLFGAVGTMISFFTISLGAIATFSRMSIGTLDVGDFLAIGAIFSATDSVCTLQVLHQDETPFLYSLVFGEGVVNDATSVVLFNAVQKIQFTHINAWTALQLIGNFLYLFSTSTLLGIGTGLITAFVLKKLYFGRHSTTRELAIMILMAYLSYMLAELFSLSGLLTVFFCGVLMSHVTWHNVTESSRTTSRHVFATLSFISETFIFLYVGMDALDFEKWKTSSLSFGGTLGVSGVLMGLVMLGRAAFVFPLSFLSNLAKKHQSEKISFRMQVVIWWAGLMRGAVSMALALNKFTRSGHTQLHGNAIMITSTITVVLFSTMVFGMITKPLIRLLLPASGHPRELSEPSSPKSFHSPLLTSQQGSDLESTTNIVRPSSLRGLLTKPTHTVHYYWRKFDDALMRPVFGGRGFVPFVPGSPTERNPPDLSKA

SEQ ID NO: 32 - protein, 545 aa, Zea mays
MSMGYQVVAAQLKVASSADHASVVIITLFVALLCACIVLGHLLEENRWLNESITALIIGLCTGGVILMTTKGKSSHVLVFSEDLFFIYLLPPIIFIAGFQVKKKQFFRNFMTITLFGAVGTMISFFTISLGAIAIFSRMNIGTLDVGDFLAIGAIFSATDSVCTLQVLHQDETPFLYSLVFGEGVVNDATSVVLFNAVQKIQITHINAEVALQVFGNFLYLFSTSTLLGIATGLITSFVLKKLYFARHSTTRELAIMMLMAYLSYMLAELFSLSGILTVFFCGVLMSHVTWHNVTESSRITSRHVFAMLSFIAETFIFLYVGTDALDFDKWKTSSLSFGGTLGVSALIMALVLLGRAAFVFPLSVLTNFSNKHENESITFKHQVIIWWAGLMRGAVSIALAFKQFTYSGVTLDPVNAAMVTNTTIVVLFTTLVFGLLTKPLIRLLMPHRHLTMLSDDSTPKSLHSPLLTSQLGSDLEEPTNIPRPSSIRGEFLTMTRTVHRYWRKFDDAFMRPMFGGRGFVPVVPGSPIERSVPQWSEEAHNKEP

SEQ ID NO: 33 - protein, 541 aa, Zea mays
MGLGVVAELVRLGVLSSTSDHASVVSINLFVALLCACIVLGHLLEENRWVNESITALIIGLCTGVVILLTTKGKSSHILVFSEDLFFIYLLPPIIFNAGFQVKKKQFFRNFMTITLFGAVGTMISFFTISLGALGLISRLNIGALELGDYLALGAIFSATDSVCTLQVLSQDETPFLYSLVFGEGVVNDATSVVVFNALQNFDITHIDAEVVFHLLGNFFYLFLLSTVLGVATGLISALVIKKLYFGRHSTDREVALMMLMAYLSYMLAELFALSGILTVFFGCIVMSHYTWHNVTESSRITTKHAFATLSFLAETFLFLYVGMDALDIDKWRSVSDTPGKSLAISSILMGLVMVGRAAFVFPLSFLSNLAKKTEHEKISWKQQVVIWWAGLMRGAVSMALAYKKFTRAGHTQVRGNAIMITSTIIVVLFSTMVFGLLTKPLINLLIPHRNATSMLSDDSSPKSLHSPLLTSQLGSDLEEPTNIPRPSSIRGEFLTMTRTVHRYWRKFDDAFMRPMFGGRGFVPFVPGSPTERNPPDLSKA

SEQ ID NO: 34 - protein, 543 aa, Gossypium hirsutum
MVAPQLAAVFTKLQTLSTSDHASVVSMNIFVALLCACIVIGHLLEENRWMNESITALIIGVFTGVIILLTSGGKSSHLLVFSEDLFFIYLLPPIIFNAGFQVKKKQFFRNFITIMLFGAVGTLISCTIISLGVINFFKEMDIGSLDIGDFLAIGAIFAATDSVCTLQVLNQDETPLLYSLVFGEGVVNDATSVVLFNAIQSFDLVNTSPRILLEFIGSFLYLFLASTMLGVIVGLVSAYIIKKLYFGRHSTDREFALMMLMAYLSYIMAELFYLSGILTVFFCGIVMSHYTWHNVTESSRVTTKHAFATLSFVAETFLFLYVGMDALDMEKWRFVSDSPGTSVAVSAVLMGLVMVGRAAFVFPLSFLSNLAKKSTSEKISFREQIIIWWAGLMRGAVSMALAYNQFTRGGHTQLRGNAIMITSTITIVLFSTVVFGLMTKPLIRFLLPHPKPTASMLSDQSTPKSMEAPFLGSGQDSFDDSLIGVHRPNSIRALLTTPAHTVHYYWRKFDNAFMRPMFGGRGFVPFVPGSPTERSEPNLPQWQ

SEQ ID NO: 35 - protein, 557 aa, Suaeda maritima
MWSQLSSFFASKMDMVSTSDHASVVSMNLFVALLCGCIVIGHLLEENRWMNESITALLIGLSTGIIILLISGGKSSHLLVFSEDLFFIYLLPPIIFNAGFQVKKKQFFRNFITIILFGAVGTLVSFIIISLGSIAIFQKMDIGSLELGDLLAIGAIFAATDSVCTLQVLNQDETPLLYSLVFGEGVVNDATSVVLFNAIQNFDLTHIDHRIAYRIAFQFGGNFLYLFFASTLLGAVTGLLSAYVIKKLYFGRHSTDREVALMMLMAYLSYMLAELFYLSGILTVFFCGIVMSHYTWHNVTESSRVTTKHAFATLSFVAEIFIFLYVGMDALDIEKWRFVSDSPGTSVAVSSILLGLLMVGRALLFSLVFLMNLSKKSNSEKVTFNQQIVIWWAGLMRGAVSVALAYNQFSRSGHTQLRGNAIMITSTITVVLFSTMVFGLLTKPLILFMLPQPKHFTSASTVSDLGSPKSFSLPLLEDRQDSEADLGNDDEEAYPRGTIARPTSLRMLLNAPTHTVHHYWRRFDDYFMRPVFGGRGFVPFVPGSPTEQSTTNLSQRT

SEQ ID NO: 36 - protein, 538 aa, Hordeum vulgare
MAFEVVAAQLARLSDALATSDHASVVSINLFVALLCACIVLGHLLEENRWLNESITALIIGLCTGVVILMTTKGKSSHVLVFSEDLFFIYLLPPIIFNAGFQVKKKQFFRNFMTITLFGAVGTMISFFTISLAAIAIFSKMNIGTLDVSDFLAIGAIFSATDSVCTLQVLNQDETPFLYSLVFGEGVVNDATSVVLFNALQNFDPNQIDAIVILKFLGNFCYLFVSSTFLGVFSGLLSAYIIKKLYIGRHSTDREVALMMLMAYLSYMLAELLDLSGILTVFFCGIVMSHYTWHNVTESSRVTTKHAFATLSFIAETFLFLYVGMDALDIEKWKFASDSPGKSIGISSILLGLVLVGRAAFVFPLSFLSNLTKKTELEKISWRQQIVIWWAGLMRGAVSIALAYNKFTRSGHTQLHGNAIMITSTITVVLFSTMLFGILTKPLIRFLLPASSNGDPSEPSSPKSLHSPLLTSMLGSDMEAPLPIVRPSSLRMLITKPTHTIHYYWRKFDDALMRPMFGGRGFVPYSPGSPTDPNVIVA

SEQ ID NO: 37 - protein, 540 aa, Hordeum brevisubulatum
MGWGLGDPPADYGSIMAVGLFVALMCICIIVGHLLEENRWMNESTTALLLGLGAGTVILFASSGKNSRLMVFSEDLFFIYLLPPIIFNAGFQVKKKQFFRNFMTITLFAVVGTLISFSIISLGAMGLISRLNIGALELGDYLALGAIFSATDSVCTLQVLSQDETPFLYSLVFGEGVVNDATSVVLFNAIQNFDLGNFSSLKFLQFIGNFLYLFGASTFLGVASGLLSAYVIKKLYFGRHSTDREVAIMMLMAYLSYMLAELLDLSGILTVFFCGIVMSHYTWHNVTESSRVTTKHAFATLSFISETFLFLYVGMDALDIEKWKIVSETYSPMKSITLSSIILALVLVARAAFVFPLSYLSNLTKKTAGEKISIRQQVIIWWAGLMRGAVSIALAYNKFAKSGHTQLPSNAIMITSTIIIVLFSTIVFGLLTKPLIRLLIPARHLTREVSALSEPSSPKSFLEQLTVNGPETDVENGVSIRRPTSLRMLLASPTRSVHHYWRKFDNAFMRPVFGGRGFVPFVPGSPTESSVPLLAHGSEN

SEQ ID NO: 38 - protein, 546 aa, Hordeum vulgare
MGPDLGALALRYTGLAVSDHDSIVAINIFIALLCGCIVFGHLLEGNRWVNESTTAIVLGLITGGVILLCTKGVNSRILIFSEDIFFIYLLPPIIFNAGFQVKKKQFFRNFATIILFGAVGTLISFVIITLGAMGLFRKLDVGPLELGDYLAIGAIFSATDSVCTLQVLNQDQAPLLYSLVFGEGVVNDATSVVLFNAIQNIDLNHFDVLVLLQLIGKFLYLFLTSTVLGVAAGLLSAYIIKKLCFARHSTDREVAIMILMAYLSYMLSMLLDLSGILTVFFCGIVMSHYTRHNVTESSRVTTKHTFATLSFIAEIFLFLYVGMDALDIDKWKLASSSPKKPIALSAVILGLVMVGRAAFVFPLSYLSNLSKKESHPKISFNQQVIIWWAGLMRGAVSIALAYNKYTTSGHTAVRVNAVMITSTIIVVLFSTMVFGLLTKPLINLLVPPRPGTAADISSQSFLDPLTASLLGSDFDVGQLTPQTNLQYLLTMPSRSVHRVWRKFDDKFMRPMFGGRGFVPFVPGSPIERSVHGPGLLGTVTEAENRS